(19) United States
(12) Patent Application Publication
(10) Pub. No.: US 2021/0369216 A1
(43) Pub. Date: Dec. 2, 2021

(54) METHOD AND DEVICE FOR COLLISION DETECTION OF RADIOTHERAPY EQUIPMENT

(71) Applicant: OUR UNITED CORPORATION, Xi'an City (CN)

(72) Inventors: Shifeng FAN, Xi'an City (CN); Jinsheng LI, Xi'an City (CN)

(73) Assignee: OUR UNITED CORPORATION, Xi'an City (CN)

(21) Appl. No.: 17/332,423

(22) Filed: May 27, 2021

(30) Foreign Application Priority Data
May 28, 2020 (CN) 202010470077.0

(51) Int. Cl. A61B 6/10; A61N 5/10; G16H 20/40; G16H 40/63

(52) U.S. Cl. CPC A61B 6/102; A61N 5/1049; A61N 2005/1074; G16H 40/63; A61N 2005/1097; G16H 20/40

(57) ABSTRACT

A method for collision detection of a radiotherapy equipment includes determining a collision-prone position of a target to be detected; and determining whether the target to be detected having a risk of collision with the radiotherapy equipment according to collision risk analysis data that is capable for reflecting a magnitude relationship between a first distance and a second distance. The first distance is a distance from the collision-prone position to a reference position of the radiotherapy equipment, and the second distance is a minimum distance between a target component of the radiotherapy equipment and the reference position. A distance between the reference position and the target component of the radiotherapy equipment is relatively constant when the target component of the radiotherapy equipment rotates around a rotation axis.

### CROSS-REFERENCE TO RELATED APPLICATION

This application claims priority to Chinese Patent Application No. 202010470077.0, field on May 28, 2020, which is incorporated herein by reference in its entirety.

### TECHNICAL FIELD

The present disclosure relates to the field of radiotherapy equipments, and in particular, to a method for collision detection of a radiotherapy equipment and devices for collision detection of a radiotherapy equipment.

### BACKGROUND

Currently, radiotherapy equipments are mainly used for radiotherapy of malignant tumors.

### SUMMARY

In a first aspect, a method for collision detection of a radiotherapy equipment is provided. The radiotherapy equipment includes a rotating frame that is capable of rotating around a rotation axis and a treatment couch. The method includes:

obtaining at least one collision-prone position of a target to be detected that is prone to collide with a target component in the radiotherapy equipment, the target to be detected includes at least one of the treatment couch, a patient on the treatment couch, a head positioning device for fixing the patient's head, and a body positioning device for fixing the patient's body; and the target component is a component that is fixed on a rotating frame and is capable of rotating with the rotating frame; and

determining whether there is a risk of collision between the target to be detected and the target component, according to collision risk analysis data that is capable of reflecting a magnitude relationship between a first distance and a second distance, the first distance is a minimum distance between each collision-prone position and a reference position of the radiotherapy equipment; and the second distance is a minimum distance between the target component and the reference position, and a distance between the reference position and the target component is relatively constant when the target component rotates around the rotation axis.

In a second aspect, a device for collision detection of a radiotherapy equipment is provided. The radiotherapy equipment includes a rotating frame that is capable of rotating around a rotation axis and a treatment couch. The device includes an obtaining device and a processing device.

The obtaining device is configured to obtain at least one collision-prone position of a target to be detected that is prone to collide with a target component in the radiotherapy equipment, the target to be detected includes at least one of the treatment couch, a patient on the treatment couch, a head positioning device for fixing the patient's head, and a body positioning device for fixing the patient's body; and the target component is a component that is fixed on the rotating frame and is capable of rotating with the rotating frame.

The processing device is configured to determine whether there is a risk of collision between the target to be detected and the target component according to collision risk analysis data that is capable for reflecting a magnitude relationship between a first distance and a second distance, the first distance is a minimum distance between each collision-prone position and a reference position of the radiotherapy equipment; the second distance is a minimum distance between the target component and the reference position; and a distance between the reference position and the target component is relatively constant when the target component rotates around a rotation axis.

In a third aspect, a device for collision detection of a radiotherapy equipment is provided. The device includes a memory and a processor. The memory is used to store computer-executable instructions, and the processor is connected to the memory. The processor executes the computer-executable instructions stored in the memory when the device for collision detection of the radiotherapy equipment is running, so that the radiotherapy equipment performs one or more steps of the method for collision detection of the radiotherapy equipment as described in the first aspect.

In a fourth aspect, a non-transitory computer-readable storage medium is provided. The non-transitory computer-readable storage medium has stored thereon computer-executable instructions. One or more steps of the method for collision detection of the radiotherapy equipment as described in the first aspect are implemented when a computer executes the computer-executable instructions.

In a fifth aspect, a computer program product for collision detection of a radiotherapy equipment is provided. The computer program product is stored on a non-transitory computer-readable storage medium, and the computer program product is configured to perform one or more steps of the method for collision detection of the radiotherapy equipment as described in the first aspect when running on a computer.

In a sixth aspect, a radiotherapy equipment is provided, including the device for collision detection of the radiotherapy equipment provided in the second or third aspect.

### BRIEF DESCRIPTION OF THE DRAWINGS

In order to describe technical solutions of the present disclosure more clearly, accompanying drawings to be used in embodiments of the present disclosure will be introduced briefly. Obviously, the accompanying drawings to be described below are merely some embodiments of the present disclosure. These accompanying drawings are only used to explain, rather than limit the embodiments of the present disclosure, and for those of ordinary skilled in the art, other drawings may be obtained based on these drawings. In addition, accompanying drawings in the following description may be regarded as schematic diagrams, and are not limitations on actual sizes of devices, actual processes of methods, etc. that the embodiments of the present disclosure relate to.

FIG. 1A is a schematic front view of a radiotherapy equipment;

FIG. 1B is a schematic top view of a treatment couch of a radiotherapy equipment;

FIG. 2A is a schematic flow diagram of a method for collision detection of a radiotherapy equipment according to the embodiments of the present disclosure;

FIG. 2B is a schematic flow diagram of another method for collision detection of a radiotherapy equipment according to the embodiments of the present disclosure;

FIG. 3 is a schematic flow diagram of yet another method for collision detection of a radiotherapy equipment according to the embodiments of the present disclosure;

FIG. 4A is a schematic cross-sectional view of a critical space of a radiotherapy equipment being cut by a target plane according to the embodiments of the present disclosure;

FIG. 4B is a schematic sectional view being cut by a P-P plane in FIG. 4A;

FIG. 40 is a schematic diagram showing that there is a target collision-prone position in collision-prone positions in a mode of body radiation therapy according to the embodiments of the present disclosure;

FIG. 5 is a schematic cross-sectional view of a critical space of another radiotherapy equipment being cut by a target plane according to the embodiments of the present disclosure;

FIG. 6 is a schematic cross-sectional view of a critical space of further radiotherapy equipment being cut by a target plane according to the embodiments of the present disclosure;

FIG. 7A is a schematic sectional view of a radiotherapy equipment in a mode of head radiation therapy according to the embodiments of the present disclosure;

FIG. 7B is a schematic cross-sectional view of a critical space corresponding to FIG. 7A being cut by a P-P plane passing through a collision-prone position and perpendicular to a rotation axis;

FIG. 8A is a schematic sectional view of another radiotherapy equipment in a mode of head radiation therapy according to the embodiments of the present disclosure;

FIG. 8B is a schematic diagram showing a combination of cross sections of a critical space corresponding to FIG. 8A that being cut by a P1-P1′ plane and a P2-P2′ plane that both pass through a collision-prone position and are perpendicular to a rotation axis;

FIG. 9A is a schematic sectional view of a radiotherapy equipment when a reference position is an isocenter according to the embodiments of the disclosure;

FIG. 9B is a schematic sectional view of a radiotherapy equipment when a reference position is a rotation axis according to the embodiments of the present disclosure;

FIG. 9C is a schematic sectional view of a radiotherapy equipment when a reference position is a circumference centered at an isocenter on an XZ plane passing through the isocenter according to the embodiments of the present disclosure;

FIG. 9D is a schematic sectional view of a radiotherapy equipment when a reference position is a cylindrical surface with a rotation axis as a central axis according to the embodiments of the present disclosure;

FIG. 10 is a schematic diagram of a critical space of a radiotherapy equipment according to the embodiments of the present disclosure.

FIG. 11 is a schematic flow diagram of yet another method for collision detection of a radiotherapy equipment according to the embodiments of the present disclosure;

FIG. 12 is a schematic flow diagram of yet another method for collision detection of a radiotherapy equipment according to the embodiments of the present disclosure,

FIG. 13 is a schematic flow diagram of yet another method for collision detection of a radiotherapy equipment according to the embodiments of the present disclosure;

FIG. 14 is a schematic flow diagram of yet another method for collision detection of a radiotherapy equipment according to the embodiments of the present disclosure;

FIG. 15 is a schematic flow diagram of yet another method for collision detection of a radiotherapy equipment according to the embodiments of the present disclosure,

FIG. 16 is a schematic diagram showing a structure of a radiotherapy equipment according to the embodiments of the present disclosure;

FIG. 17 is a schematic diagram showing a structure of a device for collision detection of a radiotherapy equipment according to the embodiments of the present disclosure; and

FIG. 18 is a schematic diagram showing a structure of another device for collision detection of a radiotherapy equipment according to the embodiments of the present disclosure.

### DETAILED DESCRIPTION

Technical solutions in the present disclosure will be described clearly and completely in combination with accompanying drawings in the present disclosure. Obviously, the described embodiments are merely some but not all of the embodiments of the present disclosure. All other embodiments obtained by a person of ordinary skill in the art on the basis of the embodiments of the present disclosure shall be included in the protection scope of the present disclosure.

The term used herein is only for the purpose of describing specific embodiments, and is not intended to limit the present disclosure. The singular forms “a”, “an” and “the” as used herein are intended to also include the plural forms, unless the context clearly indicates otherwise. It may be further understood that the terms “including” and/or “containing” when used in the description designate the existence of the described features, integers, steps, operations, elements and/or components, but do not exclude the existence or addition of one or more other features, integers, steps, operations, elements, components, and/or groups thereof.

In the description, terms such as “for example,” “as an example,” “one embodiment,” “some embodiments,” “exemplary embodiments,” “example,” “specific example” or “some examples” are intended to indicate that specific features, structures, materials or characteristics related to the embodiment(s) or the example(s) are included in at least one embodiment or example of the present disclosure. Schematic representations of the above terms do not necessarily refer to the same embodiment(s) or example(s). In addition, the specific features, structures, materials or characteristics may be included in any one or more embodiment(s) or example(s) in any suitable manner.

Hereinafter, the terms such as “first” and “second” are used for descriptive purposes only and are not to be construed as indicating or implying the relative importance or implicitly indicating the number of indicated technical features. Thus, the features defined as “first” and “second” may explicitly or implicitly include one or more of the features. In the description of the embodiments of the present disclosure, “a plurality of”, “the plurality of” and “multiple” mean two or more unless otherwise specified.

Some embodiments may be described using the expression “connected” along with its derivatives. For example, term “connected” may be used in the description of some embodiments to indicate that two or more components are in direct physical or electrical contact with each other. The embodiments disclosed herein are not necessarily limited to the content herein.

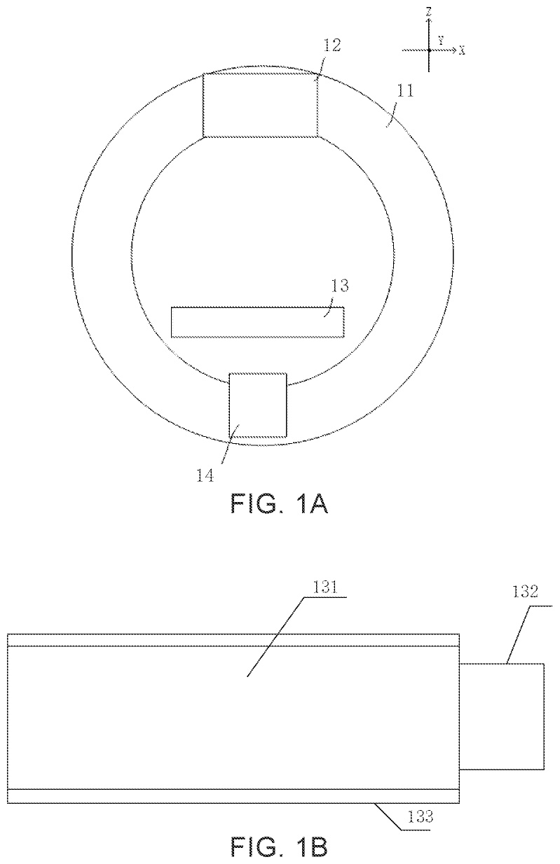

As shown in FIG. 1A, a radiotherapy equipment usually includes: a rotating frame 11 that may rotate around a rotation axis, a treatment head 12 disposed on the rotating frame, and a treatment couch 13 for transferring a patient below the treatment head 13 for radiation therapy. The rotating frame 11 may rotate 360° around the rotation axis (extending alone a Y direction in FIG. 1A) in a plane (XZ plane) perpendicular to the rotation axis. The rotation of the treatment head 12 may be driven by the rotating frame 11. In addition, the treatment head 12 may also move alone an extending direction of the rotation axis and swing in an arc. For example, the rotating frame 11 is a roller.

In addition, the radiotherapy equipment further includes an image device 14 disposed on the rotating frame 11.

In a process of radiation therapy, since the rotating frame may rotate around the rotation axis, components such as the treatment head and the image device fixed on the rotating frame will also rotate with the rotating frame 11. In addition, the treatment head itself is also able to move or swing. In this way, there is a possibility that a component fixed on the rotating frame (i.e., a subsequent target component) collides with the treatment couch or the patient when the treatment couch moves with a patient lying thereon. Such a collision may cause serious damages to the radiotherapy equipment and may also endanger personal safety of the patient on the treatment couch.

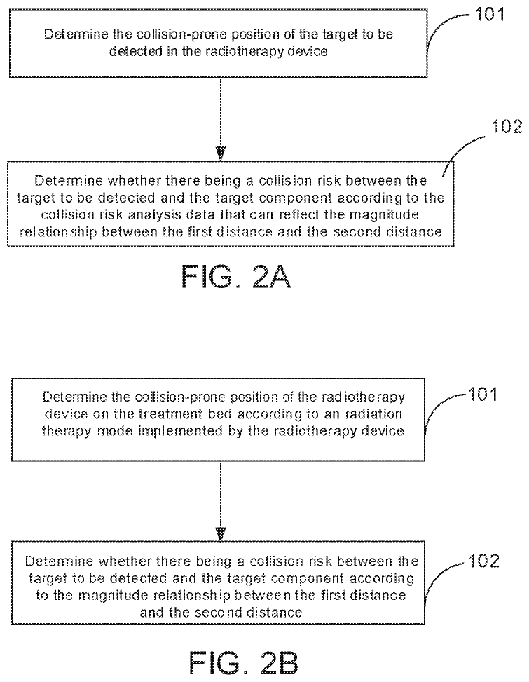

Some embodiments of the present disclosure provide a method for collision detection of a radiotherapy equipment. Referring to FIG. 2A, the method includes steps 101 and 102 (S101 and S102).

It will be noted that the technical solutions provided by the embodiments of the present disclosure are carried out without moving the treatment couch during a preparation phase (such as positioning or waiting for treatment, etc.) before the radiotherapy equipment implements any type of radiation therapy (e.g., radiotherapy to head or radiotherapy to body) according to a treatment plan. Once the treatment couch moves, for example, after the treatment couch moves back and forth or left and right during a treatment process, a target to be detected will also move accordingly. Therefore, after a position of the treatment couch is fixed again, the technical solutions provided by the embodiments of the present disclosure need to be carried out again.

In S101, at least one collision-prone position of the target to be detected that is prone to collide with a target component in the radiotherapy equipment is obtained.

The target to be detected is at least any of the following: a treatment couch, a patient on the treatment couch, a head positioning device for fixing the patient's head, and a body positioning device for fixing the patient's body. The target component is a component, in the radiotherapy equipment, that is fixed on the rotating frame and is able to rotate with the rotating frame. For example, the target component is a treatment head or an image device, and of course, it may also be another component.

The technical solutions provided by the embodiments of the present disclosure may be carried out before radiation therapy. When the technical solutions of the embodiments of the present disclosure are implemented, the target to be detected may not enter a treatment space (a space enclosed when the rotating frame rotates 360° around the rotation axis, or a space enclosed by a roller in a case where the rotating frame 11 is the roller, i.e., an inner cavity of the roller) of the radiotherapy equipment or the target to be detected may not be located at a target position in the treatment space. Therefore, the above-mentioned collision-prone position may be determined based on a mechanical structure of the radiotherapy equipment and a target position of the target to be detected in the treatment plan. Collision detection is a pre-judgment detection, that is, whether the target to be detected has interference with a moving component of the radiotherapy equipment is judged in advance when the target to be detected reaches the target position or when the target to be detected moves towards the target position, so as to avoid damages to the radiotherapy equipment and the patient. Therefore, the collision detection is the pre-judgment detection at an early stage in the entire radiation therapy process. If there may has interference, a system will issue an alarm prompt before the target to be detected enters into the treatment space of the radiotherapy equipment, thereby stopping the patient or the treatment couch from entering into the treatment space of the radiotherapy equipment.

The target to be detected is prone to collide with the target component in the radiotherapy equipment during the treatment process. That is to say, during the treatment process, there may be a risk of collision between the target to be detected and the target component of the radiotherapy equipment. Since the target to be detected needs to enter into a space enclosed by the rotating frame of the radiotherapy equipment or a space enclosed after the rotating frame rotates during the treatment process, in view of in an ordinary radiotherapy equipment, the treatment head, the image device, etc. are usually disposed on a rotating frame to facilitate treatment. Therefore, the risk of collision usually means that a risk of collision between the target to be detected and the treatment head or the image device of the radiotherapy equipment. In this case, S101 may include obtaining a collision-prone position of the target to be detected that is prone to collide with the treatment head or the image device of the radiotherapy equipment.

In some examples, the target to be detected is a treatment couch. Referring to FIG. 2B, S101 includes: obtaining at least one collision-prone position of the treatment couch of the radiotherapy equipment that is prone to collide with a target device, according to a radiation therapy mode to be implemented by the radiotherapy equipment.

For example, the radiation therapy mode includes, but is not limited to, radiation therapy to head or radiation therapy to body.

The collision-prone positions of the target to be detected depend on a specific mechanical structure of the radiotherapy equipment, and for different radiation therapy modes using the radiotherapy equipments with same mechanical structure, positions of the treatment couch and the treatment head or image device are also different, thus the collision-prone positions are also different.

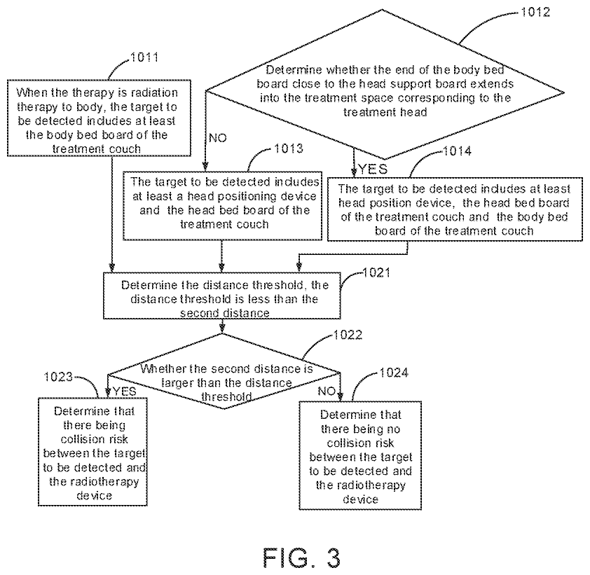

In some examples, as shown in FIG. 1B, the treatment couch 13 of the radiotherapy equipment includes a body bed board 131 for placing the patient's body, a head support board 132 for placing the patient's head, and guardrails 133 located on opposite sides of the body bed board 131 and connected to the body bed board 131 and used for preventing the patient from falling. The following takes a risk of collision between the target to be detected and the treatment head of the radiotherapy equipment as an example, referring to FIG. 3, S101 includes risks of collisions of two different radiation therapy modes, namely, a collision risk detection for the radiation therapy to body and a collision risk detection for the radiation therapy to head. The radiation therapy to head includes two situations in which the treatment couch extends below the treatment head and the treatment couch does not extend below the treatment head (i.e., steps 1013 and 1014 (S1013 and S1014)), which will be described in detail below.

In step 1011 (S1011), when the radiation therapy mode is the radiation therapy to body, at least one collision-prone position of the body bed board of the treatment couch that is prone to collide with the target component is obtained.

In this case, the target to be detected includes at least the body bed board of the treatment couch.

For example, the patient may also be fixed on the treatment couch by a body positioning device, so the target to be detected may also include the body positioning device. In this case, there is also a need to obtain a collision-prone position of the body positioning device that is prone to collide with the target component.

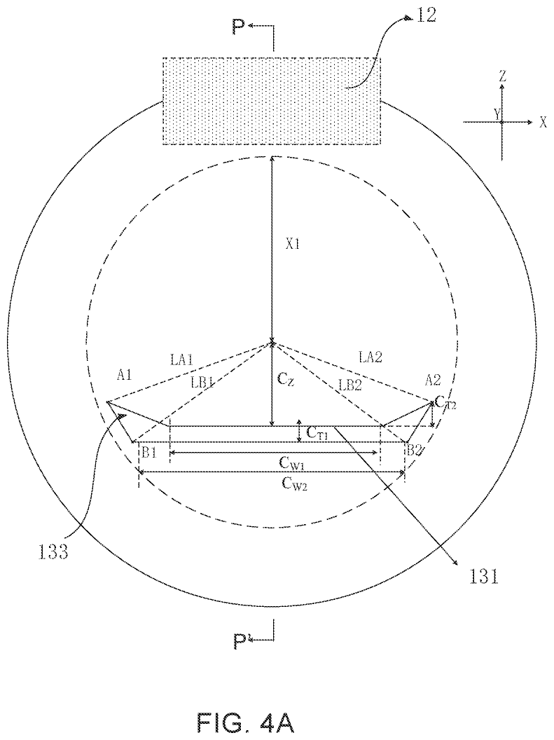

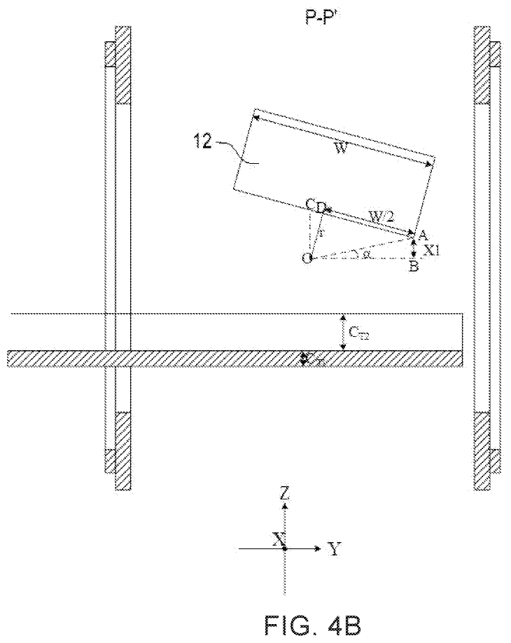

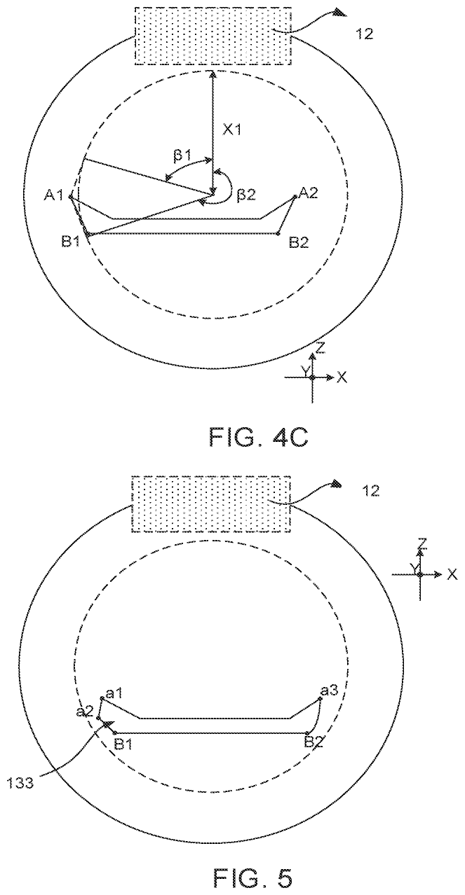

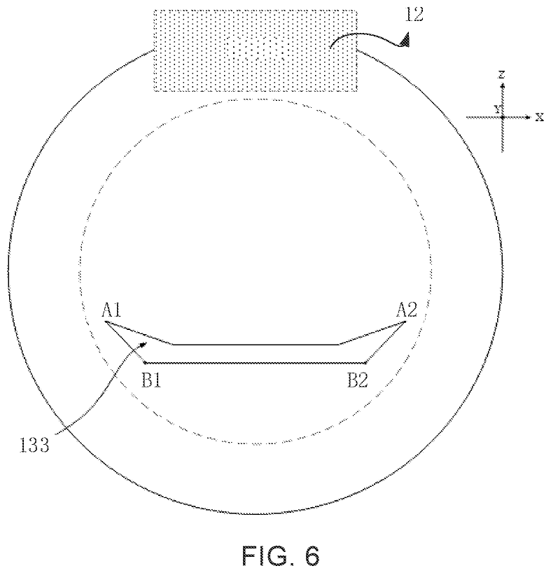

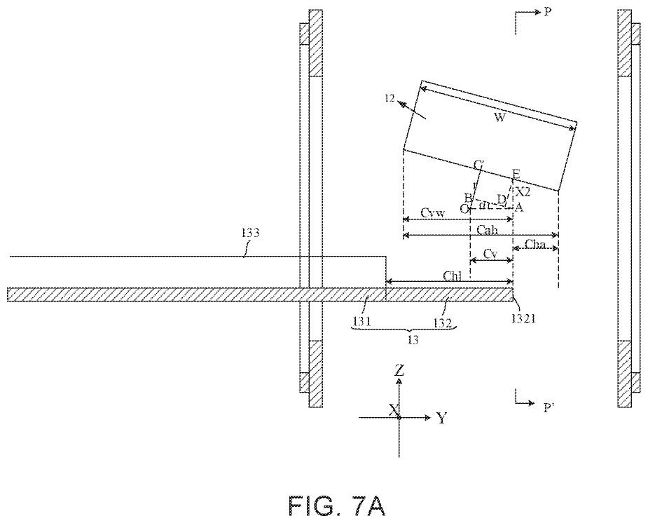

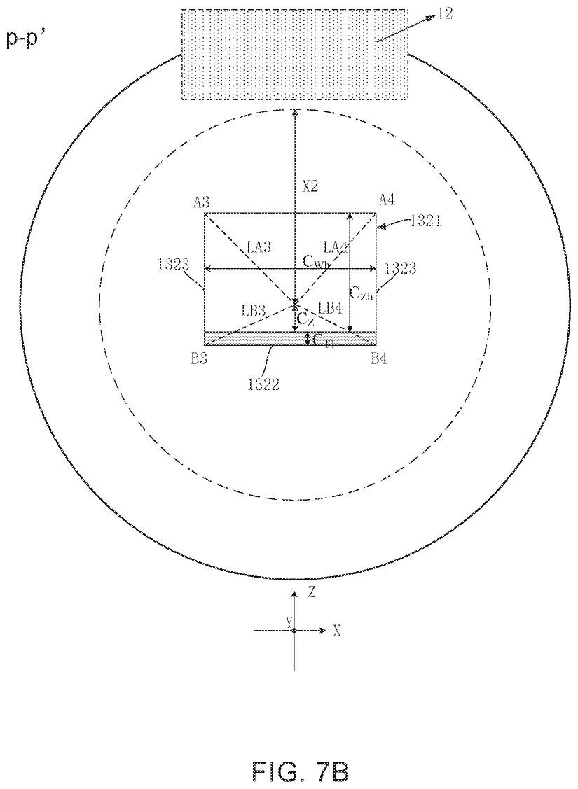

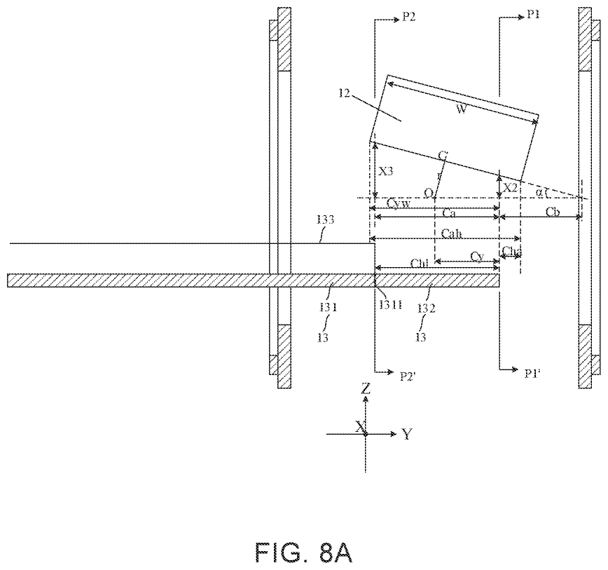

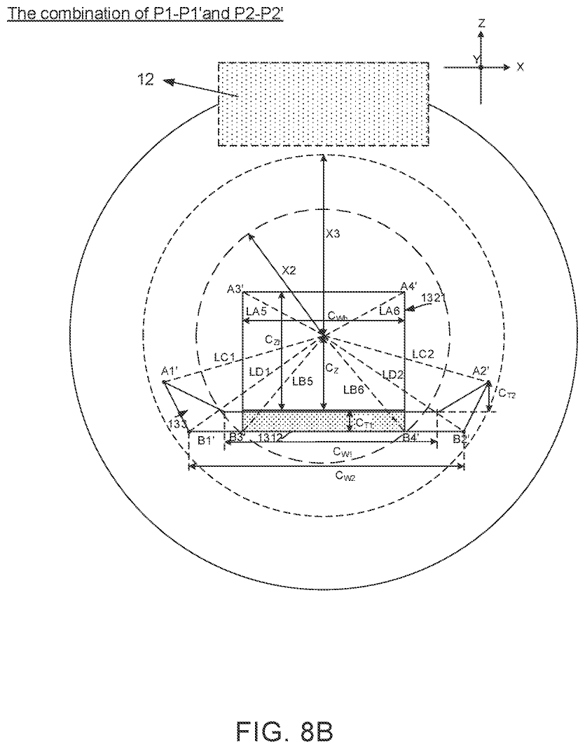

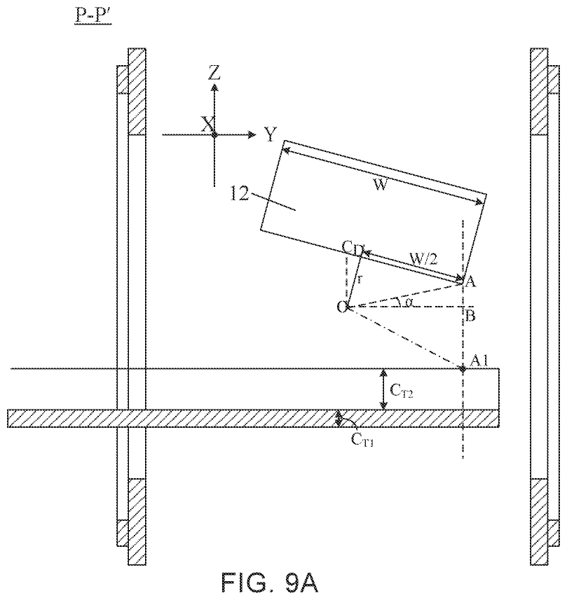

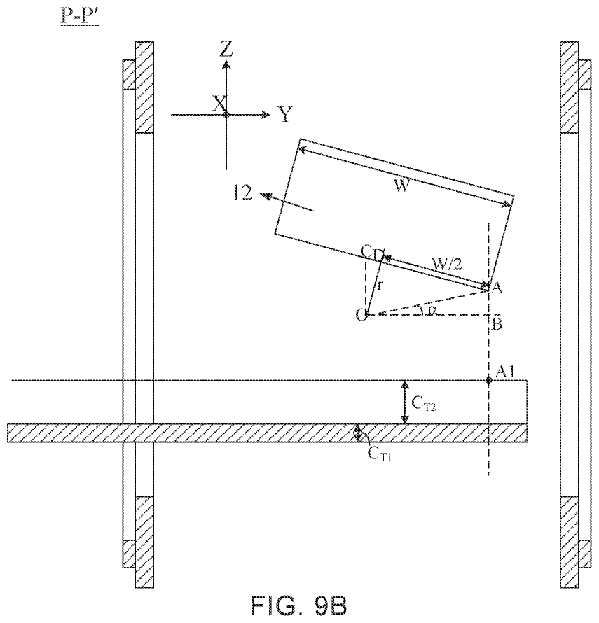

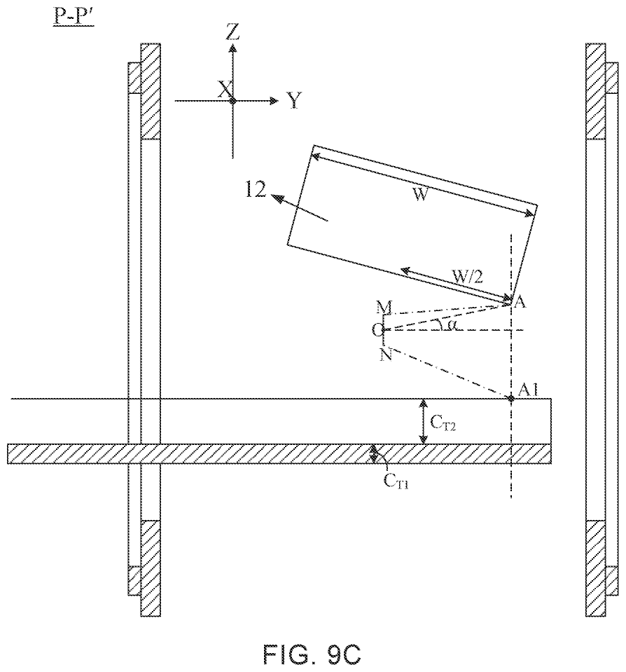

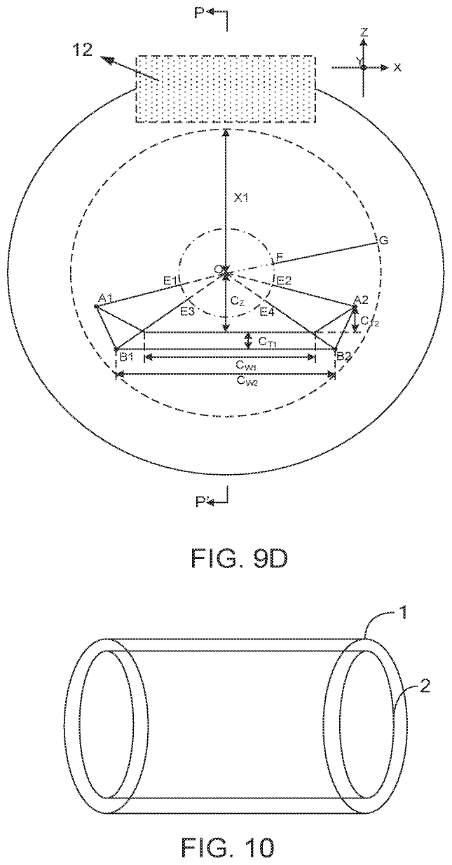

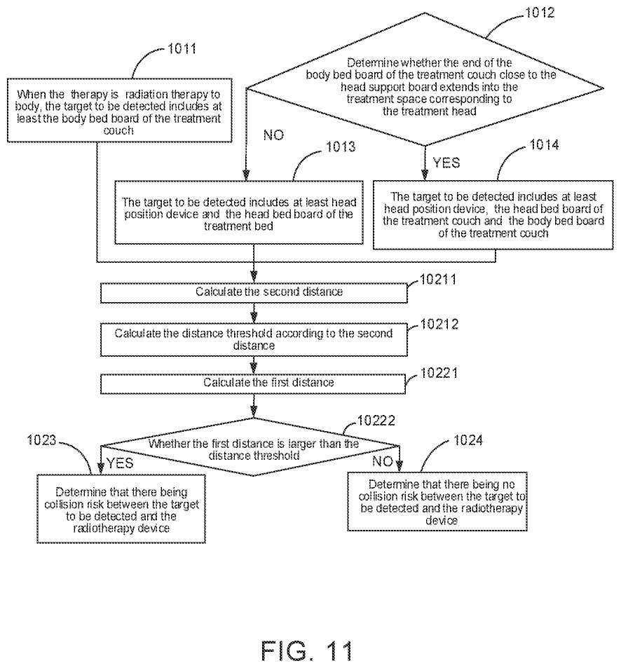

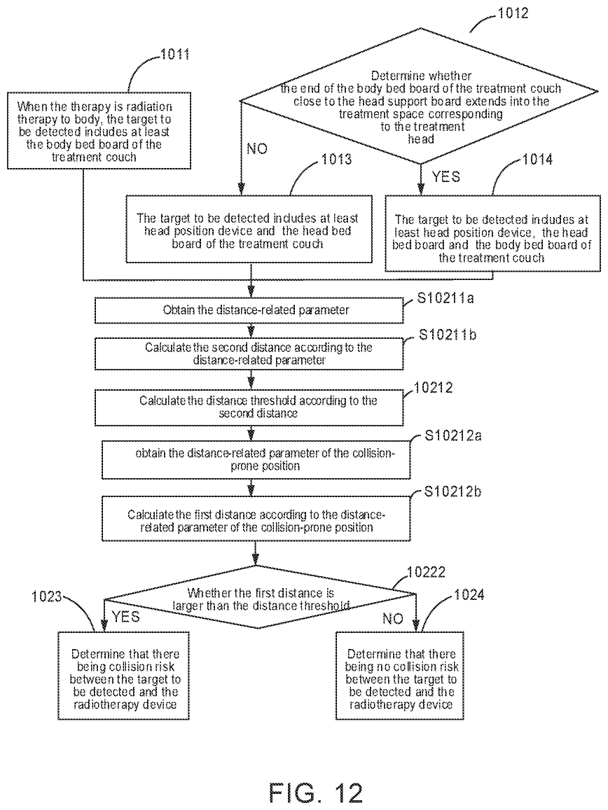

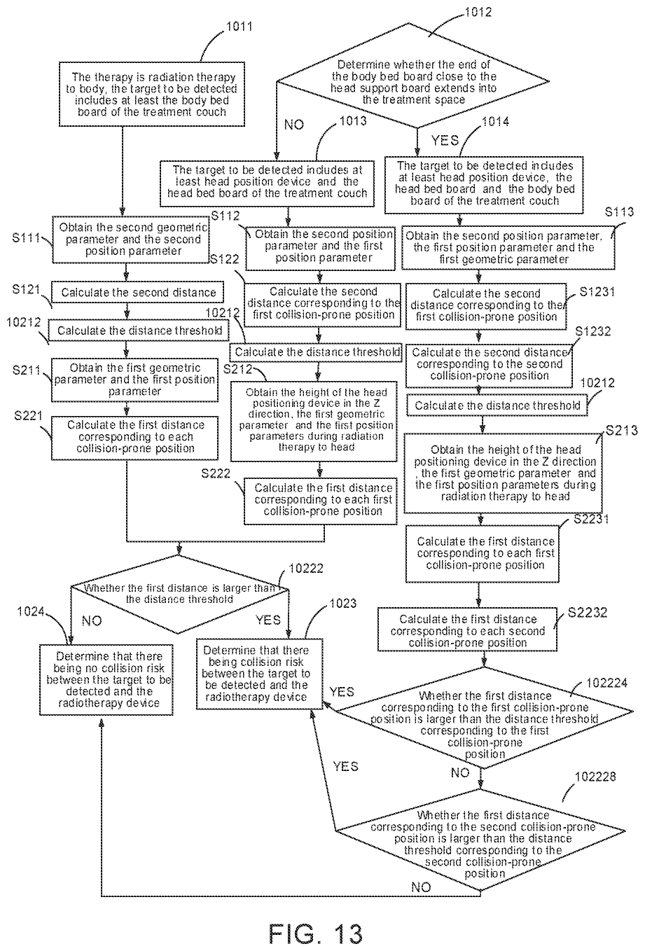

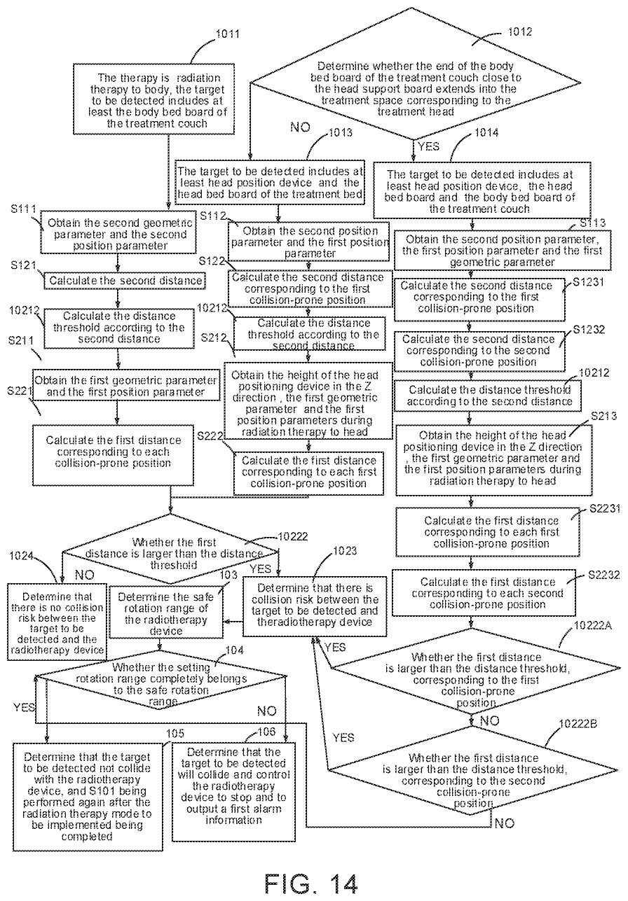

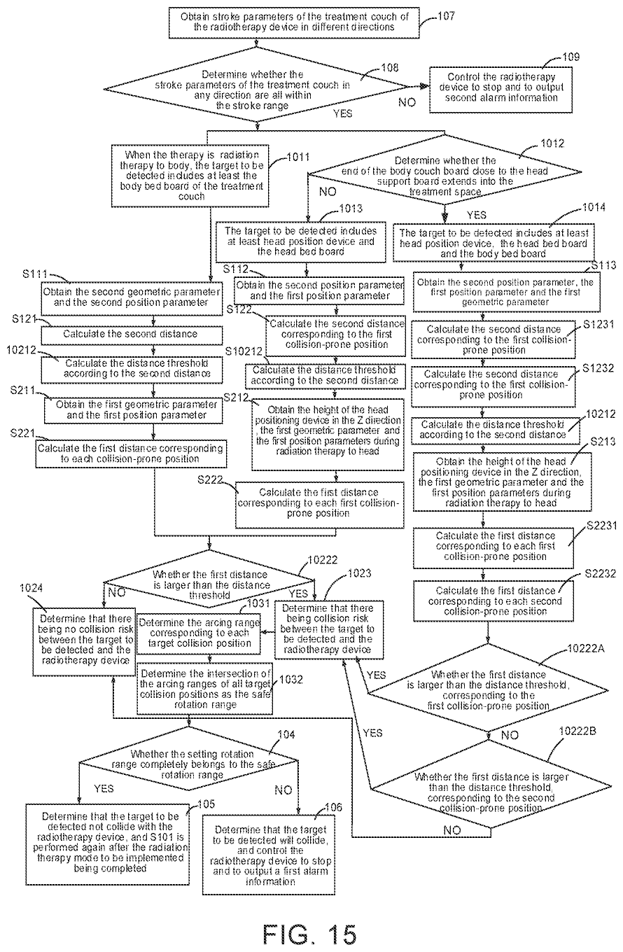

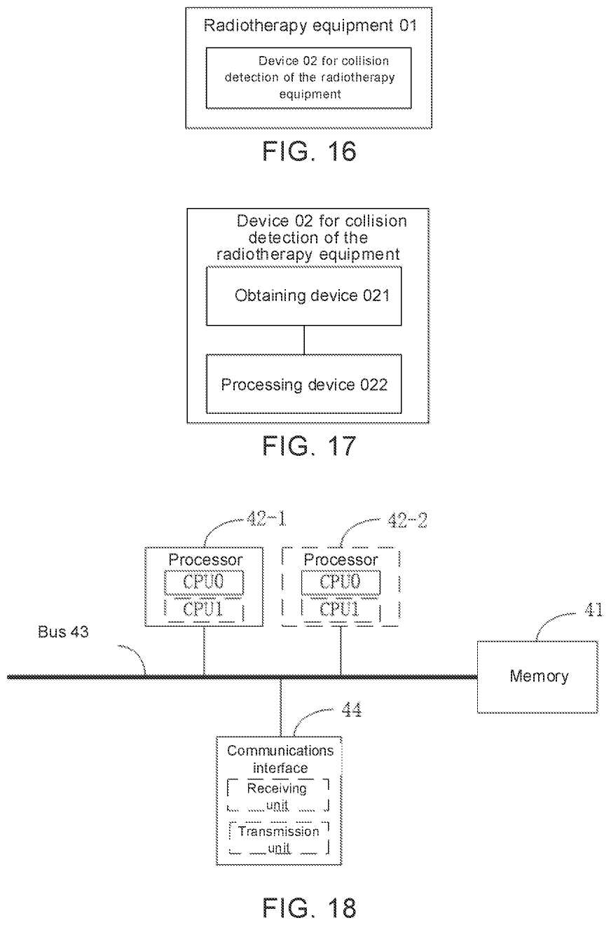

In a case where the target to be detected is of a convex polygon shape or has a convex side, since it is easy to find the collision-prone position, optionally, the collision-prone positions include corner point(s) of a cross section of the target to be detected, and the cross section is a plane being cut by a target plane (XZ plane in FIG. 4A) perpendicular to the rotation axis of the radiotherapy equipment. In the embodiments of the present disclosure, a Y direction is the extending direction of the rotation axis, a Z direction is a height direction of the treatment couch of the radiotherapy equipment, and any two of the Y direction, an X direction, and the Z direction are perpendicular to each other.

In a case where the target to be detected is the body bed board of the treatment couch, in some examples, the collision-prone positions include: in a first face of the body bed board being cut by the target plane passing through the treatment head and perpendicular to the rotation axis, two points of a bottom edge, a first target point, and a second target point. The bottom edge is a side in the first face that is farthest away from the treatment head when the treatment head rotates to a highest point of the radiotherapy equipment. The first target point is an end point, on a first lateral side of two lateral sides that are connected to the bottom edge in the first face, far away from the bottom edge, i.e., a first point closest to a boundary of a critical space. The second target point is an end point, on a second lateral side of the two lateral sides that are connected to the bottom edge in the first face, far away from the bottom edge, i.e., a second point closest to the boundary of the critical space. The critical space is a space enclosed by a radiation surface of radiotherapy rays emitted by the treatment head when the treatment head of the radiotherapy equipment rotates one cycle around the rotation axis. The critical space is located in the treatment space.

Taking the rotating frame being a roller as an example, referring to FIG. 4A, which is a schematic cross-sectional diagram of a roller-type radiotherapy equipment being cut along a target plane passing through the treatment head and perpendicular to the rotation axis. A small circle in the figure (shown as a dashed circle) is a schematic diagram of the boundary of the critical space formed by the radiation surface of the radiotherapy rays emitted by the treatment head after the treatment head rotates one cycle. For example, the first face of the body bed board 131 of the treatment couch that is cut by the target plane is a face enclosed by A1, A2, B2, and B1 connected in sequence when the radiation therapy mode to be implemented is the radiation therapy to body. When the treatment head rotates, two points B1 and B2 on a bottom edge of the first face of the body bed board 131 of the treatment couch, and a first target point A1 and a second target point A2 that are closest to the boundary of the critical space are all collision-prone positions at where collisions are most likely to occur in different directions. These four points are all corner points of the first face of the body bed board of the treatment couch that is cut by the target plane.

For example, referring to a situation shown in FIG. 5, when a section of the guardrail 133 on at least one of two sides of the body bed board 131 is in an arc shape or a zigzag shape (FIG. 5 shows that the section of guardrail 133 on one side is in a zigzag shape, and the section of the guardrail on the other side is in an arc shape), collision-prone positions where collisions are most likely to occur when the treatment couch is moving are a1, a2, a3, B1, and B2 in FIG. 5. In addition, referring to FIG. 6, when the sections of the guardrails 133 on both sides of the body bed board 131 are straight lines, if an angle between the guardrail 133 and the body bed board 131 is greater than a certain predetermined angle (for example, the predetermined angle is greater than 90°), no matter how the treatment couch moves, a collision-prone position where a collision is most likely to occur is A1 or A2 in FIG. 6. It may be seen that the collision-prone positions may be determined according to the corner points of the first face of the target to be detected being cut by the target plan passing through the treatment head and perpendicular to the rotation axis, and also according to an actual structure of the treatment couch in combination with a shape of the first face. According to actual situations, other possible corner points may also be selected as the collision-prone positions, and specific determination methods have been described above and will not be repeated here. It will be noted that a section of the treatment couch actually used is not limited to the cases shown in FIGS. 4A, 5, and 6, and the present disclosure only takes what are shown in the figures as examples for illustrative description.

The situation shown in FIG. 4A is that the treatment couch of the radiotherapy equipment is located under a central axis in the extending direction of the rotation axis (Y direction), that is, a median line of the body bed board extending in the Y direction is located under an isocenter (i.e., an isocenter of the radiotherapy equipment), which is one situation in radiation therapy. In the actual treatment process, it is also possible that the treatment couch moves in translation a certain distance to the left or a certain distance to the right, which is taken an example, and no specific restrictions are set.

In step 1012 (S1012), whether an end of the body bed board of the treatment couch that is proximate to the head support board extends into the critical space corresponding to the treatment head is determined when the radiation therapy mode is the radiation therapy to head.

If the end of the body bed board of the treatment couch that is proximate to the head support board does not extend into the critical space corresponding to the treatment head, step 1013 (S1013) is performed; and if the end of the body bed board of the treatment couch that is proximate to the head support board extends into the critical space corresponding to the treatment head, step 1014 (S1014) is performed.

In S1013, at least one collision-prone position of the head positioning device for fixing the patient's head and the head support board of the treatment couch, which is prone to collide with the target component is obtained.

For example, the collision-prone positions (referred to as first collision-prone positions) of the head support board and the head positioning device of the treatment couch are obtained when an orthographic projection of the head positioning device on the head support board all falls within a range of the head support board.

It will be noted that if the head positioning device is not used in the radiation therapy process, the targets to be detected are the head support board and the patient's head when the end of the body bed board of the treatment couch that is proximate to the head support board does not extend into the critical space corresponding to the treatment head. In this case, the collision-prone position should be located on the head support board or the patient's head. However, in actual operations, in order to reduce a risk of accidental injury to the patient, generally, the collision-prone position is not set on the patient's head. An alternative way is to simulate the patient's head as a three-dimensional virtual object (such as a cube structure) located above the head support board, and use the three-dimensional virtual object as a head positioning device to determine the collision-prone position. A shape and a size of the three-dimensional virtual object are approximately the same as a shape and a size of the patient's head. In a case where an orthographic projection of the three-dimensional virtual object on the head support board all falls within the range of the head support board, the collision-prone positions are located on the head support board and the head positioning device, and are the first collision-prone positions described above.

In an embodiment, referring to FIGS. 7A and 7B, the first collision-prone positions include: on a second face (a side face of the head support board indicated by 1321 in FIG. 7A) of the head support board away from the body bed board, two end points (B3 and B4 in FIG. 7B) on a bottom edge of the head support board and target vertexes. A rectangle is constructed with the bottom edge on the second face as a first side, and two parallel straight lines extending in the Z direction and perpendicular to the first side as second sides connected to the first side. A length of the second side is at least a maximum height of the head positioning device in the Z direction (or a maximum thickness of the patient's head corresponding to the radiotherapy equipment), and the target vertexes are two end points (A3 and A4 in FIG. 7B) of a top edge opposite to the first side, of the constructed rectangle. Of course, in actual operations, an outline of a front view of the head positioning device in the Y direction is not necessarily the above-mentioned rectangle, but the outline of the front view of the head positioning device in the Y direction is located inside the rectangle.

For example, referring to FIG. 7A, in a case where the end of the body bed board 131 of the treatment couch 13 that is proximate to the head support board 132 does not extend into the critical space corresponding to the treatment head, a length Cyw of the treatment couch 13 extending below the treatment head is less than or equal to a length Chl of the head support board 132 in the Y direction.

Cyw=Cah−Cha,Cah=W×cos α,Cha=sqrt(r×r(W/2)×(W/2))×cos(arc tan(w/(2×r))+α),

r is a distance between the treatment head and the isocenter, W is a width of the treatment head in the Y direction, and a is a swing angle of the treatment head swung along the Y direction, i.e., a non-coplanar angle. In this case, there is only a need to consider whether the head support board on which the patient's head is placed and the head positioning device (or the patient's head) have risks of collisions with the treatment head. Referring to FIG. 7B, in order to ensure that the patient's head is not collided, using a bottom edge of a side face 1321 of the head support board as a first side 1322 and using the two parallel straight lines extending in the Z direction and perpendicular to the first side as second sides 1323 connected to the first side 1322 to construct a rectangle is required. A length of the second side 1323 is at least the maximum height of the head positioning device in the Z direction including the head support board (or in the Z direction, the maximum thickness of the patient's head including the head support board). It may be concluded that A3, A4, B3, and B4 in FIG. 7B are points where collisions are most likely to occur, i.e., the collision-prone positions. In this case, the collision-prone positions are: corner points of sections when the head positioning device (or the patient's head) and the head support board of the treatment couch all being cut by the target plane perpendicular to the rotation axis. A lowest point of the treatment head according to the radiation therapy mode to be implemented is located in the target plane perpendicular to the rotation axis. The schematic cross-sectional view shown in FIG. 7B is only an example of the head support board of the treatment couch, and a central axis of the head support board is located under the isocenter. In actual operations, the position of the head support board of the treatment couch may change in the X direction and the Z direction, and the cross-sectional view shown in FIG. 7B may be adjusted appropriately according to corresponding changes.

In S1014, at least one collision-prone position of the head positioning device, the head support board of the treatment couch, and the body bed board of the treatment couch that is prone to collide with the target component is obtained.

In an embodiment, referring to FIGS. 8A and 8B, the collision-prone positions include the collision-prone positions of the head support board of the treatment couch and the head positioning device (i.e. the above-mentioned first collision-prone positions) and the collision-prone positions of the body bed board of the treatment couch (referred to as second collision-prone positions). For the determination of the first collision-prone positions may refers to the manner described above, which will not be repeated here. For determination of the second collision-prone positions, the second collision-prone positions include: two end points of a bottom edge of a third face of the body bed board that is connected to the head support board (a joint face 1311 of the body bed board 131 and the head support board 132 in FIG. 8A), a third target point, and a fourth target point. The third target point is a point which is on one side of the two sides connected to the bottom edge of the third face and closest to the boundary of the critical space, and the fourth target point is a point which is on the other side of the two sides connected to the bottom edge of the third face and closest to the boundary of the critical space.

For example, referring to FIG. 8A, in a case where the end of the body bed board 131 of the treatment couch 13 that is proximate to the head support 132 extends into the critical space corresponding to the treatment head, the length Cyw of the treatment couch 13 extending below the treatment head is greater than the length Chl of the head support board 132 in the Y direction, and Cyw is a difference between Cah and Cha (Cyw=Cah−Cha). In this case, there is a need to not only consider whether the head support board carrying the patient's head and the head positioning device (or the patient's head) have risks of collisions with the treatment head, but also consider whether a connection portion of the body bed board 131 and the head support board 132 of the treatment couch has a risk of collision with the treatment head. Therefore, the collision-prone positions in this case should include two end points B3′ and B4′ of a bottom edge of a second face 1321 of the head support board 132 away from the body bed board 131, the target vertexes A3′ and A4′, as well as two end points B1′ and B2′ of the bottom edge of the third face where the body bed board 131 and the head support board 132 are connected, and the third target point A1′ and the fourth target point A2′ that are shown in FIG. 8B (in the figure, the guardrails 133 on two sides being symmetrical with respect to the YZ plane passing through the central axis of the body bed board 131 is taken as an example, and in practice, the guardrails 133 on two sides may also be asymmetrical). Determination of the collision-prone positions of the end points B1′ and B2′, the third target point A1′, and the fourth target point A2′ may refer to the obtaining of the collision-prone positions of the body bed board 131 described above, and will not be repeated here. The schematic sectional view shown in FIG. 8B is only an example in which both central axes, in the Y direction, of the body bed board and the head support board of the treatment couch are located under the isocenter. In practice, the position of the treatment couch may change in the X direction and Z direction, and the sectional view shown in FIG. 8B may be adjusted appropriately according to the corresponding changes.

In S102, whether there is a risk of collision between the target to be detected and the target component is determined according to collision risk analysis data that can reflect a magnitude relationship between a first distance and a second distance.

The first distance is a minimum distance between each collision-prone position and a reference position of the radiotherapy equipment, and the second distance is a minimum distance between the target component and the reference position. A distance between the above-mentioned reference position and the radiotherapy equipment is constant when the rotating frame rotates around the rotation axis. The reference position includes a reference point, a reference line, or a reference face.

For example, the reference point may be the isocenter of the radiotherapy equipment; the reference line may be a central line of the rotation axis where the isocenter is located (an extending direction of the central line is the same as the extending direction of the rotation axis); the reference face may be a circumference centered at the isocenter and located on the XZ plane perpendicular to the rotation axis; or the reference face may be a circumferential section of a cylinder with the rotation axis as an axis being cut by a plane perpendicular to the rotation axis and passing through the collision-prone position. It will be noted that a radius of the circumference and a radius of a cylindrical face should be less than a radius of a circumference of the critical space being cut by the plane perpendicular to the rotation axis of the radiotherapy equipment and passing through the collision-prone position. In some embodiment, taking the risk of collision between the target to be detected and the treatment head as an example, the collision-prone position of the target to be detected is determined by a method corresponding to S1011, and the first distance and the second distance corresponding to the reference point listed above are described in the following situations.

In a first situation, referring to FIG. 9A (only a collision-prone position A1 has shown in FIG. 9A), the reference position is an isocenter O, and a first distance corresponding to the collision-prone position A1 is a distance OA1 between the isocenter O and the collision-prone position A1. The second distance is a distance from a point of the treatment head closest to the collision-prone position A1, which is located in a plane perpendicular to a rotation axis OB and passing through the collision-prone position A1, to the isocenter, i.e., a length of OA.

In a second situation, referring to FIG. 9B (only the collision-prone position A1 has shown in FIG. 91), the reference position is a central line OB (i.e., the reference line) of the rotation axis, and the first distance corresponding to the collision-prone position A1 is a distance between the collision-prone position A1 and a point which having a shortest length in the line connected to the collision-prone position A1 among any points on the central line OB. That is, the first distance is a vertical distance A1B from the collision-prone position A1 to the central line OB of the rotation axis. The second distance is a distance between a point of the treatment head closest to the collision-prone position A1 (i.e., the closest point) which is located in the plane perpendicular to the rotation axis and passing through the collision-prone position A1, and a point which having a shortest length in the line connected to the closest point among any points on the central line OB, i.e., a vertical distance AB from the closest point to the central line OB.

In a third situation, referring to FIG. 9C (only the collision-prone position A1 has shown in FIG. 9C), the reference position is the reference face, and the reference face is a circumferential face centered at isocenter and located on the XZ plane. A line segment MN passing through the isocenter O in the figure represents a side view of the reference face. The first distance corresponding to the collision-prone position A1 is a distance A1N between the collision-prone position A1 and a point which having a shortest length in the line connected to the collision-prone position A1 among any points on the reference face. The second distance is a length of a distance AM between a point of the treatment head closest to the collision-prone position A1 (i.e., the closest point), which is located in the plane perpendicular to the rotation axis and passing through the collision-prone position A1, and a point which having a shortest length in the line connected to the closest point among any points on the reference surface.

In a fourth situation, referring to FIG. 9D, FIG. 9D is a schematic sectional view of a roller-type frame (the frame has not fully shown), and a smaller circle in the figure (indicated by dashed lines) is a schematic cross-sectional view of a critical space formed by a radiation surface of radiotherapy rays emitted by a target component (such as a treatment head) after rotating one cycle, being cut by a plane perpendicular to the rotation axis and passing through the collision-prone position. The reference position is the reference face, and the reference face is a circumferential section (a smallest circle in FIG. 9D (indicated by dash-dotted lines)) of a cylinder with the rotation axis as the axis that is cut by the plane perpendicular to the rotation axis and passing through the collision-prone position. The first distance corresponding to the collision-prone position A1 is a distance A1E1 between the collision-prone position A1 and a point which having a shortest length in the line connected to the collision-prone position A1 among any points on the reference face. The second distance is a difference (e.g., a length of FG) between a radius of the smaller circle and a radius of the smallest circle in FIG. 9D. Similarly, a first distance corresponding to a collision-prone position A2 is A2E2, a first distance corresponding to a collision-prone position B1 is B1E3, and a first distance corresponding to a collision-prone position B2 is B2E4.

For example, the collision risk analysis data that can reflect the magnitude relationship between the first distance and the second distance involved in S102 includes the first distance and the second distance.

For example, the collision risk analysis data that can reflect the magnitude relationship between the first distance and the second distance involved in S102 may also be other data that are able to reflect the magnitude relationship between the first distance and the second distance indirectly. For example, in the above four situations, it may be space coordinates of two ends of line segments corresponding to the first distance and the second distance in the same coordinate system, or it may be mathematical expressions of the line segments corresponding to the first distance and the second distance respectively in the same coordinate system, and the magnitude relationship between the first distance and the second distance may be obtained by simple calculations after obtaining these data. As another example, in the above-mentioned second situation, it may be a mathematical expression of the line segment corresponding to the first distance, and the mathematical expression of a circumference with the isocenter point as the center and with the second distance as the radius, which is on the plane perpendicular to the rotation axis and passing through the collision-prone position, in the same coordinate system. For example, when it is concluded according to the mathematical expression of the line segment and the mathematical expression of the circumference, that there is an intersection point between the first distance and the second distance and the intersection point is not the end point of the line segment, it may be determined that the first distance is greater than or equal to the second distance.

In some examples, the collision risk analysis data includes the first distance and the second distance. Referring to FIGS. 2A and 2B, S102 includes: determining whether there is a risk of collision between the target to be detected and the target component according to the magnitude relationship between the first distance and the second distance.

For example, the second distance is a distance between a point of the target component closest to the collision-prone position, which is located in a plane perpendicular to the rotation axis of the radiotherapy equipment and passing through the collision-prone position, and the reference point. The target component may include at least any of the followings: a treatment head and an image device. Another example, the second distance is a distance between a point of the target component closest to the collision-prone position, which is located in the plane perpendicular to the rotation axis of the radiotherapy equipment and passing through the collision-prone position, and a point which having the shortest length in the line connected to the collision-prone position A1 among any points on the reference line or reference face.

In order to further reduce the risk of collision, the second distance which have been subtracted a rated value may be compared with the first distance, and then determining whether the target to be detected has a risk of collision with the target component of the radiotherapy equipment. Therefore, referring to FIG. 3, S102 may include steps 1021 to 1024 (S1021 to S1024).

In S1021, a distance threshold is determined, the distance threshold is obtained according to the second distance, and the distance threshold is less than the second distance.

In an example that the first distance and the second distance are obtained according to the reference line which is the central line of the rotation axis, the second distance is a minimum distance between the target component and the reference line of the radiotherapy equipment.

In order to explain why it is more secure after determining the distance threshold, a critical space corresponding to the distance threshold may be defined as a reference critical space for explanation. Taking the target component being the treatment head as an example, the reference critical space corresponding to the distance threshold is a space enclosed by a plane rotating one cycle around the rotation axis, the plane having a distance between the radiation surface of the treatment head being the distance threshold and parallel to the radiation surface and is located on a side of the radiation surface of the treatment head proximate to the rotation axis.

For example, referring to FIG. 10, the critical space enclosed by the rotation of the radiation surface of the treatment head is a space inside a cylinder 1. The reference critical space corresponding to the distance threshold is a space inside a cylinder 2, the cylinder 2 is located in the cylinder 1. The cylinder 1 and the cylinder 2 are coaxial, axes of them are the central line of the rotation axis, and a difference between radii of the cylinder 1 and the cylinder 2 is a reliable threshold Xd. Similarly, when the critical space is a space enclosed by a surface of another target component of the radiotherapy equipment towards the isocenter rotating one cycle, a relationship between the critical space enclosed by the rotation of the target component and the reference critical space corresponding to the distance threshold is similar to the situation shown in FIG. 10. It may be seen that by reducing the critical space enclosed by the rotation of the target component to the reference critical space determined by the distance threshold, it is equivalent that the critical space obtained by the rotation of the target component becomes smaller. That is, it is equivalent to translating the target component to the isocenter by a certain distance and the length of the distance is the length of the distance threshold, so that the second distance is smaller than an actual one. In this way, it is easier to determine the risk of collision between the target to be detected and the target component and to obtain existence of the risk of collision risk earlier.

In some embodiments, in an example in which the second distance is a minimum distance between the target component and the central line of the rotation axis of the radiotherapy equipment, and the target component is the treatment head, referring to FIG. 11, S1021 may include steps 10211 and 10212 (S10211 and S10212).

In S10211, the second distance is calculated.

For example, FIG. 4B shows an example in which a second distance X1 is determined in a case of the aforementioned S1011, FIG. 7A shows an example in which a second distance X2 is determined in a case of the aforementioned S1013, and FIG. 8A shows an example in which a second distance is determined in a case of the aforementioned S1014.

The second distance may be determined according to at least one of the following parameters: when the target component is at a zero position (i.e., a position of a highest point of a longitudinal axis of the circumference), a distance r from a bottom face of the target component to the reference position, a width W of the target component in the extending direction of the rotation axis, a swing angle α of the target component in a direction of the rotation axis, and a distance Cy that a front end of the treatment couch (i.e., an end of the head support board 132 away from the body bed board 131) deviates from the isocenter of the radiotherapy equipment alone the direction of the rotation axis. In subsequent description of the second distance, related parameters used to calculate the second distance are defined as distance-related parameters. As an example, referring to FIG. 12, S10211 may include steps 10211a and 10211b (S10211a and S10211b).

In S10211a, the distance-related parameters are obtained; and

in S10211b, the second distance is obtained according to the distance-related parameters.

In an example in which the target component is the treatment head and the reference position is the central line of the rotation axis, there are three different collision-prone positions on the radiotherapy equipment when a radiation therapy plan includes two different radiation therapy modes, that is, when the radiotherapy plan includes S1011, S1013, and S1014. Based on this, referring to FIG. 13, the second distance may be obtained through the following steps:

In step 111 (S111), a second geometric parameter and a second position parameter of the treatment head are obtained.

It will be noted that S111 is mainly for the radiation therapy to body, and the second geometric parameter and the second position parameter in S111 in FIG. 13 are the aforementioned distance-related parameters.

For example, referring to FIGS. 4A and 4B, when the guardrails 133 of the treatment couch are symmetrical to each other in the YZ plane through a central axis of the body bed board, the second geometric parameter may include a width W of the radiation surface of the treatment head in the Y direction; and the second position parameter may include: a swing angle α of the treatment head swinging in the direction of the rotation axis (i.e., a non-coplanar angle α of the treatment head) during the radiation therapy to body, and a distance r from a bottom face (i.e. the radiation surface) of the treatment head to the reference position (i.e., a distance from the radiation surface of the treatment head to the isocenter) when the treatment head is placed at the zero position.

In step 121 (S121), the second distance is obtained based on a third predetermined distance formula according to the second geometric parameter and the second position parameter.

In some embodiments, as can be seen from a geometric relationship in FIG. 4B that:

X1=AB=OA×cos ∠OAB;

$$OA = \sqrt{{r}^{2} + {( \frac{W}{2} )}^{2}} ;$$

∠OAB=∠COA=α+∠DOA=α+arc tan(W/2r);

CO is parallel to AB and AB is perpendicular to OB, therefore, it can be seen that the third redetermined distance formula is:

$$X 1 = \sqrt{{r}^{2} + {( \frac{W}{2} )}^{2}} \times cos ( arctan \frac{W}{2 r} + α ) ;$$

and

X1 is the second distance which is corresponding to a radius of the critical space. It will be noted that, generally, the a of the radiation therapy to body is zero in an actual treatment process.

In step 112 (S112), a second position parameter of the treatment head and a first position parameter of the treatment couch are obtained.

It will be noted that S112 is mainly for a case where the head support board does not extend into the critical space corresponding to the treatment head during the radiation therapy to head. The second position parameter and the first position parameter in S112 in FIG. 13 are also the above-mentioned distance-related parameters.

For example, referring to FIGS. 7A and 7B, the second position parameter may include: the non-coplanar angle α of the treatment head and the distance r from the bottom face (radiation surface) of the treatment head to the reference position when the treatment head is placed at the zero position (i.e., the distance r from the radiation surface of the treatment head to the isocenter) during the radiation therapy to head. The first position parameter may include the distance Cy that the front end of the treatment couch deviates from the isocenter of the radiotherapy equipment in the direction of the rotation axis (i.e., the distance between the end of the head support board away from the body bed board and a projection point of the isocenter to the head support board) during the radiation therapy to head.

In step 122 (S122), a second distance corresponding to the first collision-prone position is obtained based on a fourth predetermined distance formula according to the first position parameter and the second position parameter.

In some embodiments, referring to FIG. 7A, ED is parallel to CO and ED is perpendicular to AB, as well as AB is perpendicular to CO and AB is parallel to CE. As can be seen from a geometric relationship in FIG. 7A that:

X2=AE=ED×cos ∠DEA=ED×cos α;

a length of DE is equal to a length of CB: CB=CO−BO=r−AO×sin∠BAO=r−Cy×sin α.

It may be seen that the fourth predetermined distance formula is:

X2=(r−Cy×sin(α))/cos(α); and

X2 is the second distance corresponding to the first collision-prone position in the current embodiment.

In step 113 (S113), the second position parameter of the treatment head, the first position parameter of the treatment couch, and a first geometric parameter of the treatment couch are obtained.

It will be noted that S113 is mainly for a case where the head support board extends into the critical space corresponding to the treatment head during the radiation therapy to head. The first position parameter, the second position parameter, and the first geometric parameter in S113 in FIG. 13 are the above-mentioned distance-related parameters.

For example, referring to FIGS. 8A and 8B, the first geometric parameter may include a length Chi of the head support board in the Y direction when the guardrails 133 of the treatment couch are symmetrical to each other with respect to the YZ plane through the central axis of the body bed board; the second position parameter may include: the non-coplanar angle α of the treatment head and the distance r from the bottom face (radiation surface) of the treatment head to the reference position when the treatment head is placed at the zero position (i.e., the distance from the radiation surface of the treatment head to the isocenter) during the radiation therapy to head; and the first position parameter includes the distance Cy that the front end of the treatment couch deviates from the isocenter of the radiotherapy equipment in the direction of the rotation axis (i.e., the distance between the end of the head support board away from the body bed board and the projection point of the isocenter to the head support board) during the radiation therapy to head.

In step 1231 (S1231), the second distance corresponding to the first collision-prone position is obtained based on the fourth predetermined distance formula according to the first position parameter and the second position parameter.

For example, the manner of obtaining the second distance X2 corresponding to the first collision-prone position is the same as an manner of obtaining described in S122, and the fourth predetermined distance formula used is the same as the third predetermined distance formula, which will not be repeated here.

In step 1232 (S1232), a second distance of the second collision-prone position is calculated based on a fifth predetermined distance formula according to the first geometric parameter and the second distance corresponding to the first collision-prone position.

For example, referring to FIG. 8A, as can be seen from a geometric relationship in FIG. 8A that:

X3=X2×(Ca/Cb+1);

Ca=Chi; and

Cb=X2×cot α.

Therefore, the fifth predetermined distance formula is:

X3=X2+Chl*tan α;

X2 is the second distance corresponding to the first collision-prone position, and X3 is the second distance corresponding to the second collision-prone position.

In step 10212 (S10212), a distance threshold is obtained according to the second distance.

For example, the distance threshold may be obtained by subtracting the reliable threshold Xd from the second distance calculated aforementioned. For example, the reliable threshold may be set in a range from 1 mm to 20 mm.

Referring to FIG. 3, a collision detection may be continued when the distance threshold obtained after the performance of S1021 is less than the second distance, that is:

In step 1022 (S1022), whether the first distance is greater than the distance threshold is determined.

Step 1023 (S1023) is performed when the first distance is greater than the distance threshold has been determined: and step 1024 (S1024) is performed when the first distance is less than or equal to the distance threshold has been determined.

In some embodiments, the determination of a relationship between the first distance and the distance threshold requires pre-obtaining of the first distance. For example, in an example in which the target component is the treatment head and the reference position is the rotation axis, referring to FIG. 11, step 1022 (S1022) may include steps 10221 and 10222 (S10221 and S10222).

In step 10221 (S10221), the first distance is obtained.

For example, both the first distance and the collision-prone position are associated with their distance-related parameters respective, and therefore, referring to FIG. 12, step 10221 (S10221) further includes steps 10221a and 10221b (S10221a and S10221b).

In step 10221a (S10221a), distance-related parameters of the collision-prone position are obtained: and

in step 10221b (S10221b), the first distance is obtained according to the distance-related parameters of the collision-prone position.

For example, in an example in which the target component is the treatment head and the reference position is the rotation axis, there are three different collision-prone positions on the radiotherapy equipment when the radiotherapy plan includes two different radiation therapy modes, that is, when the radiotherapy plan includes step 1011 (S1011), step 1013 (S1013), and step 1014 (S1014). Therefore, referring to FIG. 13, the method of collision detection of the radiotherapy equipment includes the following steps.

In step 211 (S211), the first geometric parameter and the first position parameter of the treatment couch are obtained.

It will be noted that step 111 (S111) is mainly for the radiation therapy to body, and the first geometric parameter and the first position parameter in step 211 (S211) in FIG. 13 are also the above-mentioned distance-related parameters.

For example, referring to FIGS. 4A and 4B, the first geometric parameter may include: a thickness C_{T1} of the body bed board, a distance C_{T2} from an upper edge of the guardrail 133 to an upper surface of the body bed board, a width C_{w1} of the upper surface of the body bed board in the X direction, and a width C_{w2} of a lower surface of the body bed board away from the isocenter in the X direction. The first position parameter may include a distance Cz between an upper surface of the body bed board proximate to the isocenter and the rotation axis, as well as a displacement Cx of the treatment couch in the X direction relative to the rotation axis of the treatment couch under the isocenter (Cx is 0 in FIGS. 4A and 4B, and as an example, it is defined in the present disclosure that Cx moving towards a positive X direction is positive, and Cx moving towards a negative X direction is negative) during the radiation therapy to body.

In step 221 (S221), a first distance corresponding to each collision-prone position is determined based on a first predetermined distance calculation formula according to the first position parameter and the first geometric parameter.

For example, referring to FIGS. 4A and 4B, when the two end points of the bottom edge of the first face of the treatment couch being cut by the target plane are B1 and B2, the first target point is A1 and is located on an upper edge of the guardrail on one side of the body bed board, as well as the second target point is A2 and is located on an upper edge of the guardrail on the other side of the body bed board, the first predetermined distance calculation formula includes: a calculation formula of A1, a calculation formula of A2, a calculation formula of B1, and a calculation formula of B2. As can be seen from a geometric relationship in FIG. 4A that the calculation formula of A1 is:

$$LA 1 = \sqrt{{( Cz - {C}_{T 2} )}^{2} + {( \frac{{C}_{w 1}}{2} - Cx )}^{2}} ;$$

as can be seen from the geometric relationship in FIG. 4A that the calculation formula of A2 is:

$$LA 2 = \sqrt{{( Cz - {C}_{T 2} )}^{2} + {( \frac{{C}_{w 1}}{2} + Cx )}^{2}} ;$$

as can be seen from the geometric relationship in FIG. 4A that the calculation formula of B1 is:

$$LB 1 = \sqrt{{( Cz + {C}_{T 1} )}^{2} + {( \frac{{C}_{w 2}}{2} - Cx )}^{2}} ;$$

as can be seen from the geometric relationship in FIG. 4A that the calculation formula of B2 is:

$$LB 2 = \sqrt{{( Cz + {C}_{T 1} )}^{2} + {( \frac{{C}_{w 2}}{2} + Cx )}^{2}} ;$$

and

LA1 is a first distance corresponding to A1, LA2 is a first distance corresponding to A2, LB1 is a first distance corresponding to B1, and LB2 is a first distance corresponding to B2.

It will be noted that, CT2 in the above-mentioned formulas for LA1 and LA2 needs to be changed according to actual situations when the guardrails on the two sides are asymmetrical to each other with respect to the YZ plane penetrating the central axis of the body bed board.

In step 212 (S212), a height of the head positioning device including the head support board in the Z direction (or a thickness of the patient's head in the Z direction including the head support board), the first geometric position of the treatment couch, and the first position parameter of the treatment couch during the radiation therapy to head are obtained.

It will be noted that step 212 (S212) is mainly for a case where the head support board does not extend into the critical space corresponding to the treatment head during the radiation therapy to head. The first geometric parameter and the first position parameter described in step 212 (S212) in FIG. 13 are also the above-mentioned distance-related parameters. Since the collision-prone position is on the head positioning device or the patient's head in the process of treatment, the position parameter of the collision-prone position is related to the height of the head positioning device in the Z direction (or the thickness of the patient's head in the Z direction), and the second geometric parameter and the second position parameter of the treatment couch.

For example, referring to FIGS. 7A and 7B, the height of the head positioning device including the head support board in the Z direction (or the thickness of the patient's head including the head support board in the Z direction) is C_{zh} during the radiation therapy to head, the first geometric parameter may include: a width C_{wh} of the head support board in the X direction and the thickness C_{T1} of the body bed; and the first position parameter may include: the distance Cz between the upper surface of the body bed board proximate to the isocenter and the rotation axis, as well as the displacement Cx of the treatment couch in the X direction relative to the rotation axis of the treatment couch located under the isocenter (Cx is 0 in FIGS. 7A and 7B, and as an example, it is defined in the present disclosure that Cx moving towards the positive X direction is positive, and Cx moving towards the negative X direction is negative) during the radiation therapy to body.

In step 222 (S222), the first distance corresponding to each first collision-prone position is obtained in the radiation therapy to head, based on a second predetermined distance calculation formula according to the height of the head positioning device including the head support board in the Z direction (or the thickness of the patient's head including the head support board in the Z direction), the first position parameter and the first geometric parameter.

For example, referring to FIG. 7B, when the two end points of the bottom edge which is away from the treatment head on the second face of the head support board away from the body bed board of the treatment couch are B3 and B4, and the target vertexes are A3 and A4, the second predetermined distance calculation formula includes: a calculation formula of A3, a calculation formula of A4, a calculation formula of B3, and a calculation formula of B4. As can be seen from a geometric relationship in FIG. 7B that the calculation formula of A3 is:

$$LA 3 = \sqrt{{( Czh - Cz )}^{2} + {( \frac{{C}_{wh}}{2} - Cx )}^{2}} ;$$

as can be seen from the geometric relationship in FIG. 7B that the calculation formula of A4 is:

$$LA 4 = \sqrt{{( Czh - Cz )}^{2} + {( \frac{{C}_{wh}}{2} + Cx )}^{2}} ;$$

as can be seen from the geometric relationship in FIG. 7B that the calculation formula of B3 is:

$$LB 3 = \sqrt{{( Cz + {C}_{T 1} )}^{2} + {( \frac{{C}_{wh}}{2} - Cx )}^{2}} ;$$

as can be seen from the geometric relationship in FIG. 7B that the calculation formula of B4 is:

$$LB 4 = \sqrt{{( Cz + {C}_{T 1} )}^{2} + {( \frac{{C}_{wh}}{2} + Cx )}^{2}} ;$$

and

LA3 is a first distance corresponding to A3, LA4 is a first distance corresponding to A4, LB3 is a first distance corresponding to B3, and LB4 is a first distance corresponding to B4.

In step 213 (S213), the height of the head positioning device including the head support board in the Z direction (or the thickness of the patient's head including the head support board in the Z direction), the first geometry parameter of the treatment couch and the first position parameter of the treatment couch are obtained during the radiation therapy to head.

It will be noted that step 213 (S213) is mainly for a case where the head support board extends into the critical space corresponding to the treatment head during the radiation therapy to head. The height of the head positioning device including the head support board in the Z direction (or the thickness of the patient's head including the head support board in the Z direction), the first geometric parameter and the first position parameter are the above-mentioned distance-related parameters of the collision-prone position during the radiation therapy to head in step 213 (S213) in FIG. 13. It will be noted that, in this embodiment, the collision-prone position is on the head positioning device or the patient's head, and therefore, the position parameters of the collision-prone position are related to the height of the head positioning device in the Z direction (or the thickness of the patient's head in the Z direction), the second geometric parameter and the second position parameter of the treatment couch.

For example, referring to FIGS. 8A and 8B, when the guardrails 133 are symmetrical to each other with respect to the YZ plane through the central axis of the body bed board, the height of the head positioning device including the head support board in the Z direction (or the thickness of the patient's head including the head support board in the Z direction) is C_{zh} during the radiation therapy to head; the first geometric parameter may include: the length Chl of the head support board in the Y direction, the thickness C_{T1} of the body bed board, the width C_{wh} of the head support board in the X direction, the distance C_{T2} from the upper edges of the guardrails on both sides of the body bed board to the upper surface of the body bed board, the width C_{w1} of the upper surface of the body bed board in the X direction, and the width C_{w2} of the lower surface of the body bed board away from the isocenter in the X direction; and the first position parameter may include: the distance Cz between the upper surface of the body bed board proximate to the isocenter and the rotation axis, as well as the displacement Cx of the rotation axis of the treatment couch in the X direction relative to the rotation axis of the treatment couch under the isocenter during the radiation therapy to head (Cx is 0 in FIGS. 8A and 8B, and as an example, it may be defined in the present disclosure that Cx moving towards the positive X direction is positive, and Cx moving towards the negative X direction is negative).

In step 2231 (S2231), the first distance corresponding to each first collision-prone position is obtained during the radiation therapy to head based on the second predetermined distance calculation formula according to the height of the head positioning device including the head support board in the Z direction (or the thickness of the patient's head including the head support board in the Z direction), the first position parameter and the first geometric parameter.

For example, referring to FIG. 8B, when the two end points of the bottom edge which is away from the treatment head, on the second face of the head support board away from the body bed board of the treatment couch are B3′ and B4′, and the target vertexes are A3′ and A4′, the second predetermined distance calculation formula includes: a calculation formula of A3′, a calculation formula of A4′, a calculation formula of B3′, and a calculation formula of B4′;

as can be seen from a geometric relationship in FIG. 8B that the calculation formula of A3′ is:

$$LA 5 = \sqrt{{( Czh - Cz )}^{2} + {( \frac{{C}_{wh}}{2} - Cx )}^{2}} ;$$

as can be seen from the geometric relationship in FIG. 8B that the calculation formula of A4′ is:

$$LA 6 = \sqrt{{( Czh - Cz )}^{2} + {( \frac{{C}_{wh}}{2} + Cx )}^{2}} ;$$

as can be seen from the geometric relationship in FIG. 8B that the calculation formula of B3 is:

$$LB 5 = \sqrt{{( Cz + {C}_{T 1} )}^{2} + {( \frac{{C}_{wh}}{2} - Cx )}^{2}} ;$$

as can be seen from the geometric relationship in FIG. 8B that the calculation formula of B4′ is:

$$LB 6 = \sqrt{{( Cz + {C}_{T 1} )}^{2} + {( \frac{{C}_{wh}}{2} + Cx )}^{2}} ;$$

and

LA5 is a first distance corresponding to A3, LA6 is a first distance corresponding to A4′, LB5 is a first distance corresponding to B3′, and LB6 is a first distance corresponding to B4′.

In step 2232 (S2232), a first distance corresponding to each second collision-prone position is obtained based on the first predetermined distance calculation formula according to the first position parameter and the first geometric parameter.

For example, referring to FIG. 8B, when the two end points of the bottom edge which is away from the treatment head, on a third face where the body bed board and the head support board are connected are B1′ and B2′, the third target point is A1′ and is located on the upper edge of the guardrail on one side of the body bed board, the fourth target point is A2 and is located on the upper edge of the guardrail on the other side of the body bed board, and the first predetermined distance calculation formula includes: a calculation formula of B1′, a calculation formula of B2′, a calculation formula of A1′, and a calculation formula of A2′;

as can be seen from the geometric relationship in FIG. 8B that the calculation formula of B1′ is:

$$LC 1 = \sqrt{{( Cz - {C}_{T 2} )}^{2} + {( \frac{{C}_{w 1}}{2} - Cx )}^{2}} ;$$

as can be seen from the geometric relationship in FIG. 8B that the calculation formula of B2′ is:

$$LC 2 = \sqrt{{( Cz - {C}_{T 2} )}^{2} + {( \frac{{C}_{w 1}}{2} + Cx )}^{2}} ;$$

as can be seen from the geometric relationship in FIG. 8B that the calculation formula of A1′ is:

$$LD 1 = \sqrt{{( Cz + {C}_{T 1} )}^{2} + {( \frac{{C}_{w 2}}{2} - Cx )}^{2}} ;$$

as can be seen from the geometric relationship in FIG. 8B that the calculation formula of is:

$$LD 2 = \sqrt{{( Cz + {C}_{T 1} )}^{2} + {( \frac{{C}_{w 2}}{2} + Cx )}^{2}} ;$$

and

LC1 is a first distance corresponding to B1′, LC2 is a first distance corresponding to B2′, LD1 is a first distance corresponding to A1′, and LD2 is a first distance corresponding to A2.

It will be noted that, CT2 in the above formulas for LC1 and LC2 needs to be changed according to actual situations when the guardrails on two sides are asymmetrical to each other relative to the YZ plane though the central axis of the body bed board.

It will be noted that the above-mentioned step 111 (S111), step 121 (S121), step 211 (S211), and step 221 (S221) correspond to the aforementioned step 1011 (S1011). As well as, step 111 (S112), step 122 (S122), step 212 (S212), and step 222 (S222) correspond to the aforementioned step 1013 (S1013). Step 113 (S113), step 123 (S123) (step 1231 (S1231) and step 1232 (S1232)), step 213 (S213), and step 223 (S223) (step 2231 (S2231) and step 2232 (S2232)) correspond to the aforementioned step 1014 (S1014).

In step 10222 (S10222), whether the first distance is greater than the distance threshold is determined.

Step 1023 (S1023) is performed when the determination of the first distance is greater than the distance threshold; and Step 1024 (S1024) is performed when the determination of the first distance is less than or equal to the distance threshold. It will be noted that the second distance is reduced to the distance threshold for comparison at this time, so there may be or may not be a determined risk of collision when the first distance and the distance threshold are equal. Therefore, it may be that step 1023 (S1023) is performed, or it may also be that step 1024 (S1024) is performed. Here, only step 1024 (S1024) is performed as an example. In a case where the first distance is used directly to compare with the second distance, step 1023 (S1023) is performed when the first distance and the second distance are equal, because a critical state should also be regarded as a special situation of the collision (that is, the target to be detected and the target component is intersecting).

Optionally, referring to FIG. 13, when S10211a and S10211b included in S10211 are S111 and S121 or are S112 and S122, and S10221a and S10221b included in S10221 are S211 and S221 or are S212 and S222, the content of S10222 remains unchanged; and when S10211a and S10211b included in S10211 are S113 and S123 (S1231 and S1232), and S10221a and S10221b included in S10221 are S213 and S223 (S2231 and S2232), S10222 includes steps 10222A, 10222B, and 10222C (S10222A, S10222B, and S10222C)

In step 10222A (S10222A), whether a first distance corresponding to the first collision-prone position is greater than a distance threshold corresponding to the first collision-prone position is determined.

The step 1023 (S1023) is performed when the first distance corresponding to the first collision-prone position is greater than the distance threshold corresponding to the first collision-prone position has been determined; and the step 10222B (S10222B) is performed when the first distance corresponding to the first collision-prone position is less than or equal to the distance threshold corresponding to the first collision-prone position has been determined.

The distance threshold corresponding to the first collision-prone position is obtained by subtracting the reliable distance threshold Xd from the second distance corresponding to the first collision-prone position.

It will be noted that, since the second distance is reduced to be in a range of the distance threshold for comparison at this time, in a case where the first distance corresponding to the first collision-prone position and the distance threshold corresponding to the first collision-prone position are equal, there may be or may not be the determined risk of collision, therefore the step 1023 (S1023) may be performed, or the step 10222B (S10222B) may also be performed. Here, only performing the step 10222B (S10222B) as an example for description.

In a case where the first distance corresponding to the first collision-prone position is directly used to compare with the second distance corresponding to the first collision-prone position, when the first distance corresponding to the first collision-prone position and the second distance corresponding to the first collision-prone position are equal, the step 1023 (S1023) is performed because of the critical state should also be regarded as a special situation of the collision (that is, the target to be detected and the target component is intersecting).

In step 10222B (S10222B), whether a first distance corresponding to the second collision-prone position is greater than a distance threshold corresponding to the second collision-prone position is determined.

The step 1023 (S1023) is performed when the first distance corresponding to the second collision-prone position is greater than the distance threshold corresponding to the second collision-prone position has been determined; and the step 1024 (S10240 is performed when the first distance corresponding to the second collision-prone position is less than or equal to the distance threshold corresponding to the second collision-prone position has been determined.

The distance threshold corresponding to the second collision-prone position is obtained by subtracting the reliable distance threshold Xd from the second distance corresponding to the second collision-prone position.

It will be noted that since the second distance is reduced to be in the range of the distance threshold for comparison at this time, in a case where the first distance corresponding to the second collision-prone position and the distance threshold corresponding to the second collision-prone position are equal, there may be or may not be the determined risk of collision, therefore the step 1023 (S1023) may be performed, or the step 1024 (S1024) may also be performed. Here, only performing the step 1024 (S1024) as an example. In a case where the first distance corresponding to the second collision-prone position is directly used to compare with the second distance corresponding to the second collision-prone position, when the first distance corresponding to the second collision-prone position and the second distance corresponding to the second collision-prone position are equal, the step 1023 (S1023) should be performed because of the critical state should also be regarded as a special situation of collision (that is, the target to be detected and the target component is intersecting).

In addition, specific contents of the above-mentioned step 10222A (S10222A) and step 10222B (S10222B) may be exchanged.

In step 1023 (S1023), there is a risk of collision between the target to be detected and the radiotherapy equipment is determined.

In step 1024 (S1024), there is no risk of collision between the target to be detected and the radiotherapy equipment is determined.

It will be noted that, in practice that the distance threshold may not be determined, that is, the magnitude relationship between the first distance and the second distance is directly determined. In this case, step 1021 (S1021) is removed and the distance threshold in step 1022 (S1022) is changed into the second distance at the same time.

Optionally, since it is assumed that the radiotherapy equipment rotates a cycle during the determination of the risk of collision of the radiotherapy equipment, and a rotation range of the radiotherapy equipment will be set in actual operations, the further determination of whether the collision really occurs is also required. Therefore, referring to FIG. 14, after step 1023 (S1023), steps 103, 104, 105 and 106 (S103, S104, S105, and S106) are also included.

In step 103 (S103), a safety rotation range of the radiotherapy equipment is determined.

As an example, referring to FIG. 15, the step 103 (S103) includes steps 1031 and 1032 (S1031 and S1032).

In step 1031 (S1031), an arc range corresponding to each target collision position is determined.

A plurality of collision-prone positions are included in the at least one target collision position, and the target collision position is a collision-prone position having a first distance greater than a corresponding distance threshold among the plurality of collision-prone positions. The radiotherapy equipment will not collide with the target collision position when the radiotherapy equipment is located in the arc range corresponding to the target collision position, the distance threshold is obtained according to the second distance and the distance threshold is less than the second distance.

As an example, step 1032 (S1032) may include: determining a critical circumference with the rotation axis as an axis and a distance threshold as a radius; constructing tangent lines to the critical circumference from each target collision positions to form tangent points respectively, and determining a larger of two central angles corresponding to the target collision position as the arc range of the target collision position.

For example, in an example in which the radiation therapy to body is the radiation therapy mode to be implemented, the reference position is the center of the rotation axis, and the second distance is the minimum distance between the target component of the radiotherapy equipment and the central line of the rotation axis, referring to FIG. 4C, A1, A2, B1, and B2 are collision-prone positions. A1 is the target collision position which the corresponding first distance is greater than the corresponding distance threshold, X1 is the second distance which is corresponding to distance threshold, the small circle is a cross section of the critical space corresponding to the distance threshold being cut by a plane passing through A1 and perpendicular to the rotation axis, the cross section is the critical circumference, and the large circle is formed by the frame of the radiotherapy equipment rotates one cycle. For example, assuming that 131 is 70° and ρ2 is 240°, the arc range of A1, i.e., a range of larger central angle corresponding to the arc is [−70°, 240°]; in the present disclosure, it is defined that a rotation angle of the radiotherapy equipment is 0° when the treatment head is right above the critical circumference (i.e., in the positive Z direction); and of course, there may be other limited manners, as long as the rotation angle and the range can be expressed.

In step 1032 (S1032), an intersection of the arc ranges of all target collision positions being a safe rotation range is determined.

For example, assuming that there are three target collision positions which corresponding arc ranges are [−70°, 210°], [0°, 230°], and [−60°, 170°] respectively, and the safe rotation range is [0°, 170°].

In step 104 (S104), whether a set rotation range of the radiotherapy equipment completely belongs to the safe rotation range is determined.

The step 105 (S105) is performed when the set rotation range completely belongs to the safe rotation range has been determined, and the step 106 (S106) is performed when the set rotation range does not completely belong to the safe rotation range has been determined.

For example, in a case where the safe rotation range is [0°, 170°] and the set rotation range is [20°, 200°], it indicates that there is only a part of the intersection between these two, that is, the set rotation range of the radiotherapy equipment in the radiation therapy mode to be implemented does not completely belong to the safe rotation range; and in a case where the safe rotation range is [0°, 170°] and the set rotation range is [20°, 150°], it indicates that the latter of these two is completely included in the former, that is, the set rotation range of the radiotherapy equipment in the radiation therapy mode to be implemented completely belongs to the safe rotation range.

In step 105 (S105), the target to be detected will not collide with the radiotherapy equipment has been determined, and step 101 (S101) is performed again after the radiation therapy mode to be implemented is completed.

In step 106 (S106), the target to be detected will collide has been determined and the radiotherapy equipment being controlled to stop and to output a first alarm information; and the first alarm information at least indicates that the radiotherapy equipment will collide.

For example, the first alarm information may be sent through a sound and light alarm, or it may be sent directly to an operation terminal corresponding to an operator of the radiotherapy equipment. The operator will reformulate a treatment plan according to the first alarm information after observing the first alarm information.

For example, the first alarm information may also carry the safety rotation range of the treatment head in the radiation therapy mode currently implemented obtained in the above-mentioned steps.

As an example, optionally, referring to FIG. 15, the method of collision detection of the radiotherapy equipment may also include steps 107, 108, and 109 (S107, S108, and S109).

In step 107 (S107), stroke parameters of the treatment couch of the radiotherapy equipment in different directions are obtained.

In step 108 (S108), whether a stroke parameter of the treatment couch in any direction is within a predetermined stroke range is determined.

For example, the stroke parameters of the treatment couch in different directions may include at least: coordinates of the X direction, coordinates of the Y direction, and coordinates of the Z direction.

The step 109 (S109) is performed when the stroke parameter of the treatment couch in any direction is not within the predetermined stroke range has been determined; and the step 101 (S101) is performed after step 108 (S108) when the stroke parameters of the treatment couch in different directions are all within the predetermined stroke range has been determined.

For example, a predetermined movement range of the X direction may be [−195 mm, 195 mm], a predetermined movement range of the Y direction may be [0, 1930 mm], and a predetermined movement range of the Z direction may be [−300 mm, 0].

In step 109 (S109), the radiotherapy equipment is controlled to stop and to output second alarm information; and the second alarm information may at least be used to indicate that the treatment couch of the radiotherapy equipment moves beyond a range.

For example, the second alarm information may be “1001: negative movement in X axis beyond range” or “1002: positive movement in X axis beyond range” when a movement parameter is not in the predetermined movement range of the X direction; the second alarm information may be “1003: negative movement in Y axis beyond range” or “1004: positive movement in Y axis beyond range” when the movement parameter is not in the predetermined movement range of the Y direction; and the second alarm information may be “1005: negative movement in Z axis beyond range” or “1006: positive movement in Z axis beyond range” when the movement parameter is not in the range of the predetermined movement of the Z direction.

Steps 107, 108 and 109 (S107, S108 and S109) may be performed before or after any step in the method for collision detection of the radiotherapy equipment provided by the embodiments of the present disclosure. As an example, FIG. 15 only shows an example of performing step 107, 108 and 109 (S107, S108 and S109) before step 101 (S101); and of course, when step 108, 109 or 110 (S108, S109 or S110) is performed after or before a certain determination step, steps subsequently performed of the relevant determination step need to be changed accordingly, and no specific limitation will be made here.

It will be noted that, first of all, the radiotherapy equipment used for examples in the above embodiments is a three-dimensional radiotherapy equipment, in practice, it may also be applied to a radiotherapy equipment of more dimensions (e.g., a four-dimensional radiotherapy equipment) or a radiotherapy equipment of less dimensions (e.g., a two-dimensional radiotherapy equipment), and only selections of the collision-prone positions and corresponding formulas need to be modified accordingly:

secondly, the accompanying drawings in the above embodiments only illustrate a case where the non-coplanar angle of the treatment head of the radiotherapy equipment rotates in a positive direction of Y, but the technical solutions provided by the embodiments of the present disclosure are not limited thereto, and when the treatment head may also rotate in a negative direction of Y, only parameters in the above formulas and the selections of the collision-prone positions need to be adjusted in a simple way; and

thirdly, the first geometrical parameter, the second geometrical parameter, the first position parameter, and the second position parameter mentioned in the above embodiments of the present disclosure may be obtained from a pre-treatment plan, or may also be derived from known quantities in the treatment plan. For example, the above-mentioned Cx and Cz may be calculated according to the known three-dimensional coordinates (X, Y, and Z directions) of the treatment couch in the treatment plan. There is no specific limitation on a method of obtaining the above parameters and any method may be used to obtain the above parameters.

For example, the parameters in the above embodiments may be as shown in Table 1 below:

TABLE 1

| Geometric parameters | Numerical value |
|---|---|
| W | 600 mm |
| r | 150 mm |
| α | 0-35° |
| C_{w1} | 500 mm |
| C_{w2} | 500 mm |
| C_{wh} | 350 mm |
| C_{zh} | 250 mm |
| C_{T1} | 40 mm |
| C_{T2} | 40 mm |
| Cx | Amount of calculation (calculated by |
| | coordinates of X, Y and Z of the treatment |
| | couch) |
| Cy | Amount of calculation (calculated by |
| | coordinates of X, Y and Z of the treatment |
| | couch) |
| Cz | Amount of calculation (calculated by |
| | coordinates of X, Y and Z of the treatment |
| | couch) |
| Xd | 1 mm-20 mm (calculated by coordinates of |
| | X, Y and Z of the treatment couch) |
| X1, X2, X3 | Amount of calculation (calculated according |
| | to the formula in the above embodiments) |
| Cah | Calculated value |
| Cyw | Calculated value |
| Chl | 420 mm |

The method for collision detection of the radiotherapy equipment provided by the embodiments of the present disclosure may include: determining the collision-prone position of the target to be detected; the target to be detected being at least any of the followings: the treatment couch, the head positioning device for fixing the patient's head, the body positioning device for fixing the patient's body; and determining whether there is a risk of collision between the target to be detected and the radiotherapy equipment according to the collision risk analysis data that can reflect the magnitude relationship between the first distance and the second distance; the first distance being the distance between the collision-prone position and the reference position of the radiotherapy equipment, and the second distance being the minimum distance between the target component of the radiotherapy equipment and the reference position of the rotation axis, and the distance between the reference position and the radiotherapy equipment is relatively constant when the radiotherapy equipment rotates around the rotation axis. In the technical solutions provided by the embodiments of the present disclosure, the collision-prone position of each device or patient (i.e., the target to be detected) which a collision with the radiotherapy equipment is most likely to occur during the treatment process may be determined first, and then whether there is a risk of collision between the target to be detected and the radiotherapy equipment may be determined according to the collision risk analysis data that can reflect the magnitude relationship of the first distance and the second distance. Since the first distance is the distance from the collision-prone position to the reference position of the radiotherapy equipment, the second distance is the minimum distance between the target component of the radiotherapy equipment and the reference position of the rotation axis, and the magnitude relationship between these two distances directly determines whether the radiotherapy equipment will collide with the collision-prone position (that is, whether the radiotherapy equipment has a risk of collision) during a rotation of the radiotherapy equipment around the rotation axis, it may be determined whether there is the risk of collision between the target to be detected and the radiotherapy equipment based on data that can reflect the first distance and the second distance. Therefore, the technical solutions provided by the embodiments of the present disclosure may determine whether there is the risk of collision in the treatment process before the radiotherapy equipment treats the patient. If it is determined that there is the risk of collision, the operator may change the radiation therapy plan in time to improve safety of radiotherapy. In order to well implement the method for collision detection of the radiotherapy equipment provided by the above embodiments, referring to FIG. 16, the embodiments of the present disclosure further provide a radiotherapy equipment 01. The radiotherapy equipment 01 may include a device 02 for collision detection of the radiotherapy equipment.

In some implementations, referring to FIG. 17, the device 02 for collision detection of the radiotherapy equipment provided by the embodiments of the present disclosure may include: an obtaining device 021 and a processing device 022. The obtaining device 021 may perform S101 (including the above-mentioned steps that may be included in S101) in the method for collision detection of the radiotherapy equipment provided by the above embodiments of the present disclosure. The processing device 022 may perform S102 (including the above-mentioned steps included in S102), S103 (including the above-mentioned steps included in S103), S104, S105, S106, S108, and S109 in the method for collision detection of the radiotherapy equipment provided by the above embodiments of the present disclosure. Of course, the obtaining device 021 and the processing device 022 may also cooperatively perform all the steps in the method for collision detection of the radiotherapy equipment provided by the above embodiments of the present disclosure. For example, the steps of obtaining information are all completed by the obtaining device 021, and the steps of determining or processing the obtained information and controlling the radiotherapy equipment are completed by the processing device 022, which will not be limited herein, as long as the obtaining device and the processing device may cooperate to implement the method for collision detection of the radiotherapy equipment provided by the above embodiments.

For example, the obtaining device 021 is configured to obtain at least one target collision position of the target to be detected that is prone to collide with the target component in the radiotherapy equipment, the target to be detected being at least any of the followings: the treatment couch, the patient on the treatment couch, the head positioning device for fixing the patient's head, and the body positioning device for fixing the patient's body; and the target component being a component that is fixed on the rotating frame and can rotate with the rotating frame in the radiotherapy equipment.

The processing device 022 may be configured to determine whether the target to be detected has a risk of collision with the target component according to the collision risk analysis data that can reflect the magnitude relationship between the first distance and the second distance, the first distance being the minimum distance between each collision-prone position and the reference position of the radiotherapy equipment; the second distance being the minimum distance between the target component and the reference position; and the distance between the reference position and the target component of the radiotherapy equipment being relatively constant when the target component of the radiotherapy equipment rotates around the rotation axis.

For example, the collision risk analysis data may include: a first distance and a second distance. The processing device 022 may be further configured to determine whether the target to be detected has a risk of collision with the radiotherapy equipment according to the magnitude relationship between the first distance and the second distance.

For example, the processing device 022 may be further configured to determine a distance threshold according to the second distance. The distance threshold is less than the second distance. There is the risk of collision between the target to be detected and the radiotherapy equipment is determined when the first distance is greater than the distance threshold; and there is no risk of collision between the target to be detected and the radiotherapy equipment is determined when the distance is less than the distance threshold.

For example, the minimum distance from the radiotherapy equipment to the reference position is the minimum distance from the target component of the radiotherapy equipment to the reference position; and the target component may include at least any of the followings: a treatment head and an image device.

For example, the target to be detected is at least any of the followings: the treatment couch, the patient on the treatment couch, the head positioning device for fixing the patient's head, and the body positioning device for fixing the patient's body.

For example, the processing device 022 may be further configured to obtain the minimum distance from the target component to the reference position according to at least one of the following parameters: the distance from the bottom face of the target component to the reference position when the target component is placed at the zero position, the width of the target component in the direction of the rotation axis, the swing angle of the target component in the direction of the rotation axis, and the distance that the front end of the treatment couch deviates from the isocenter of the radiotherapy equipment in the direction of the rotation axis.

For example, the collision-prone position may include: a corner point of a cross section of the target to be detected being cut by a plane perpendicular to the rotation axis of the radiotherapy equipment.

For example, the obtaining device 021 may be further configured to: determine the collision-prone position of the target to be detected according to the radiation therapy mode to be implemented by the radiotherapy equipment; and the radiation therapy mode may be at least any of the followings: radiation therapy to head and radiation therapy to body.

For example, in the case where the treatment couch includes a body bed board for placing the patient's body and a head support board for placing the patient, the obtaining device 021 may be further configured to: determine that the target to be detected includes at least the body bed board of the treatment couch when the radiation therapy mode is the radiation therapy to body; and determine that the target to be detected includes at least the head positioning device for fixing the patient's head and the head support board of the treatment couch when the radiation therapy mode is the radiation therapy to head.

Further, for example, in the case where the radiation therapy mode is the radiation therapy to head, the obtaining device 021 may be further configured to: determining whether an end of the body bed board of the treatment couch proximate to the head support board extends into a critical space corresponding to the treatment head. The critical space is formed when a radiation surface of the treatment head rotates one cycle around the rotation axis; and if it is determined that the end of the body bed board of the treatment couch proximate to the head support board extends into the treatment space corresponding to the treatment head, the target to be detected further includes the body bed board of the treatment couch.

For example, after the processing device 022 determines that there is the risk of collision between the target to be detected and the radiotherapy equipment, the processing device 022 may be further configured to: determining the safety rotation range of the radiotherapy equipment; if the set rotation range of the radiotherapy equipment completely belongs to the safety rotation range, it is determined that the target to be detected will not collide with the radiotherapy equipment; and if the set rotation range of the radiotherapy equipment does not completely belong to the safety rotation range, it is determined that the target to be detected will collide with the radiotherapy equipment.

For example, the processing device 022 may be further configured to: determining the arc range corresponding to each target collision position, the target collision position being a collision-prone position with the corresponding first distance greater than the corresponding distance threshold among the collision-prone positions, and the radiotherapy equipment not colliding with the target collision position when the radiotherapy equipment is within the arc range corresponding to the target collision position; and determining the intersection of the arc ranges of the target collision positions as the safety rotation range.

Further, for example, the processing device 022 may be further configured to: determining the critical circumference with the rotation axis as the axis and the distance threshold as the radius: determining the larger of these two central angles corresponding the target collision position as the arc range of the target collision position by constructing tangent lines to the critical circumference from the target collision positions to form tangent points respectively.

For example, the obtaining device 021 may be further configured to obtain the stroke parameters of the treatment couch in different directions before determining the collision-prone position of the target to be detected on the radiotherapy equipment; and to obtain the collision-prone position of the target to be detected on the radiotherapy equipment when determining that the stroke parameter of the treatment couch in any direction is within the range of the stroke parameters.

The device for collision detection of the radiotherapy equipment provided by the embodiments of the present disclosure may include: an obtaining device 021 configured to determine a collision-prone position of a target to be detected, and the target to be detected is at least any of the followings: the treatment couch, the patient on the treatment couch, the head positioning device for fixing the patient's head, and the body positioning device for fixing the patient's body.

For example, the processing device 022 is configured to determine whether there is a risk of collision between the target to be detected and the radiotherapy equipment based on the collision risk analysis data that can reflect the magnitude relationship between the first distance and the second distance. The first distance is the distance from the collision-prone position determined by the obtaining device to the reference position of the radiotherapy equipment, the second distance is the minimum distance between the target component of the radiotherapy equipment and the reference position, and the distance between the reference position and the radiotherapy equipment is constant when the radiotherapy equipment rotates around the rotation axis.

Therefore, in the technical solutions provided by the embodiments of the present disclosure, the collision-prone position which each device or patient, i.e., the target to be detected is most likely to collide with the radiotherapy equipment during the treatment process may be determined first, and then whether there is the risk of collision between the target to be detected and the radiotherapy equipment is determined based on the collision risk analysis data that can reflect the magnitude relationship of the first distance and the second distance. Since the first distance is the distance from the collision-prone position to the reference position of the radiotherapy equipment, and the second distance is the minimum distance between the target component of the radiotherapy equipment and the reference position of the rotation axis, and the magnitude relationship between these two distances directly determines whether the radiotherapy equipment will collide with the collision-prone position during the rotation of the radiotherapy equipment around the rotation axis, whether there is the risk of collision between the target to be detected and the radiotherapy equipment may be determined based on data that can reflect the first distance and the second distance. Therefore, the technical solutions provided by the embodiments of the present disclosure may determine whether there is the risk of collision in the treatment process before the radiotherapy equipment treats the patient. If it is determined that there is the risk of collision, the operator may change the radiation therapy plan in time to improve the safety of radiation therapy.

Referring to FIG. 18, the embodiments of the present disclosure further provide another device for collision detection of a radiotherapy equipment. The device includes a memory 41, a processor 42, and optionally a bus 43 and a communications interface 44. The memory 41 is used to store computer-executable instructions, and optionally, the processor 42 and the memory 41 are connected via the bus 43. When the device for collision detection of the radiotherapy equipment is operating, the processor 42 executes the computer-executable instructions stored in the memory 41 to cause the device for collision detection of the radiotherapy equipment to perform the method for collision detection of the radiotherapy equipment as provided in the above embodiments.

In an embodiment, the processor 42 (42-1 and 42-2) may include one or more central processing units (CPUs), for example, a CPU0 and a CPU1 shown in FIG. 18. Moreover, as one embodiment, the device for collision detection of the radiotherapy equipment may include a plurality of processors 42, such as a processor 42-1 and a processor 42-2 shown in FIG. 18. Each CPU of these processors may be a single-CPU, also may be a multi-CPU. The processor 42 herein may refer to one or more devices, circuits, and/or processing cores for processing data (e.g. computer program instructions).

The memory 41 may be a read-only memory (ROM) or another type of static storage device that may store static information and instructions, a random access memory (RAM) or another type of dynamic storage device that may store information and instructions, or an electrically erasable programmable read-only memory (EEPROM), a compact disc read-only memory (CD-ROM), or another compact disc storage, an optical disc storage (including compressed discs, laser discs, optical discs, digital versatile discs, and Blue-ray discs), a magnetic disk storage medium or another magnetic storage device, or any other medium that can be used to carry or store a desired program code in a form of instructions or data structures and can be accessed by a computer, which is not limited thereto. The memory may be separate and coupled to the processor 42 via the bus 43. The memory 41 may also be integrated with the processor 42.

In an embodiment, the memory 41 is used to store data in the present disclosure and execute computer-executable instructions corresponding to a software program of the present disclosure. The processor 42 may be used to implement various functions of the device for collision detection of the radiotherapy equipment by running or executing the software program stored in the memory 41 and calling data stored in the memory 41.

The communications interface 44 may use any device such as a transceiver to communicate with other devices or communications networks, such as a control system, a radio access network (RAN), and a wireless local area network (WLAN). The communications interface 44 may include a receiving unit to implement a receiving function and a transmission unit to implement a transmission function.

The bus 43 may be an industry standard architecture (ISA) bus, a peripheral component interconnect (PCI) bus, or an extended industry standard architecture (EISA) bus, etc. The bus 43 may be divided into an address bus, a data bus, a control bus, etc. For convenience of representation, only one thick line is used to represent the bus in FIG. 18, but it does not mean that there is only one bus or one type of bus.

The embodiments of the present disclosure further provide a non-transitory computer-readable storage medium. The computer-readable storage medium includes computer-executable instructions. In a case of running on a computer, the computer-executable instructions cause the computer to perform the method for collision detection of the radiotherapy equipment provided by the above embodiments of the present disclosure.

The embodiments of the present disclosure further provide a computer program. The computer program may be directly loaded into the memory and contains a software code. After being loaded by a computer and executed, the computer program may implement the method for collision detection of the radiotherapy equipment provided by the above embodiments of the present disclosure.

The embodiments of the present disclosure further provide a computer program product for collision detection of a radiotherapy equipment. The computer program product is stored on a computer-readable medium, and the computer program product is configured to perform, when running on a computer, the method for collision detection of the radiotherapy equipment provided by the above embodiments of the present disclosure.

Those skilled in the art should be aware that, in one or more of the above embodiments, the functions described in the present disclosure may be implemented by hardware, software, firmware, or any combination thereof. When implemented by software, these functions may be stored in a computer-readable medium or transmitted as one or more instructions or codes on the computer-readable medium. The computer-readable medium may include a computer-readable storage medium and a communication medium. The communication medium includes any medium that facilitates the transfer of a computer program from one place to another. The storage medium may be any available medium that may be accessed by a general-purpose or special-purpose computer.

Through the description of the above embodiments, those skilled in the art may clearly understand that, for the convenience and brevity of description, only the division of the above-mentioned functional modules is used as an example. In practical applications, the above functions may be allocated to different functional modules according to requirements. That is, the internal structure of the apparatus may be divided into different functional modules to complete all or part of the functions described above.

In several embodiments provided in the present disclosure, it will be understood that the disclosed apparatus and method may be implemented through other methods. For example, the device embodiments described above are merely illustrative, for example, the division of modules or units is only a logical functional division, and there may be other division manners in practical implementations. For example, multiple units or components may be combined or integrated into another device, or some features may be ignored, or not executed. In addition, the shown or discussed mutual coupling or direct coupling or communication connection may be indirect coupling or communication connection through some interfaces, devices or units, or may be of electrical, mechanical or other forms. The units described as separate components may be or may not be physically separated, and the component displayed as units may be one physical unit or multiple physical units, that is, the component(s) may be located in one position, or may be distributed at many different positions. Some or all of the units may be selected according to actual needs to achieve the purpose of the solutions of the embodiments.

In addition, various functional units in the embodiments of the present disclosure may be integrated into one processing unit, or may exist separately and physically, or two or more units may be integrated into one unit. The above-mentioned integrated unit may be implemented in a form of hardware or software functional unit. When the integrated unit is implemented in the form of the software functional unit and sold or used as an independent product, it may be stored in a readable storage medium. Based on this understanding, the technical solutions of the embodiments of the present disclosure or a part of the technical solutions that contribute to the prior art, or all or part of the technical solutions may be embodied in a form of software product. The software product is stored in a storage medium and includes many instructions to enable a device (such as a single-chip microcomputer or a chip) or a processor to execute all or part of the steps in the method in the embodiments of the present disclosure. The foregoing storage media include: a U disk, a mobile hard disk, a ROM, a RAM, a magnetic disk or an optical disk and other media that may store program codes.

In addition, those skilled in the art should be aware that the flow diagrams and block diagrams in the accompanying drawings may illustrate the architecture, functions, and operations of possible implementations of systems, methods, and computer program products according to various embodiments of the present disclosure. In this regard, each block in the flow diagram or block diagram may represent a part of the module, the program segment, or the code, and part of the module, program segment, or code includes one or more executable instructions for implementing (one or more) specific logic function(s). It will also be noted that, in some alternative implementations, the functions marked in the block may also occur in a different order from the order marked in the drawings. For example, two blocks shown in succession may actually be performed substantially simultaneously, or, depending on the functions involved, and the blocks may sometimes be performed in a reverse order. It will also be noted that each block in the illustration of the block diagram and/or flow diagram and the combination of the blocks in the illustration of the block diagram and/or flow diagram may be implemented by a dedicated hardware-based system that performs specified functions or actions, or it may be realized by a combination of dedicated hardware and computer instructions.

The corresponding structures, materials, behaviors, and equivalents of all methods or steps in the appended claims plus functional elements are intended to include any structure, material, or behaviors for performing functions in combination with other claimed elements. For the purpose of illustration and description, the description of the present disclosure has been presented, but the description is not meant to be exhaustive or limited to the present disclosure in the disclosed form. Many modifications and changes are apparent to those of ordinary skill in the art without departing from the scope and spirit of the present disclosure. Although some exemplary embodiments have been described in the present disclosure, the protection scope of the present disclosure is not limited thereto, and the changes or replacements that any person skilled in the art can readily conceive of within the technical scope disclosed by the present disclosure should be within the protection scope of the present disclosure. Therefore, the protection scope of the present disclosure shall be subjected to the protection scope of the claims.

What is claimed is:

1. A method for collision detection of a radiotherapy equipment, the radiotherapy equipment including a rotating frame that is capable of rotating around a rotation axis and a treatment couch, the method comprises:
obtaining at least one collision-prone position of a target to be detected that is prone to collide with a target component in the radiotherapy equipment, wherein the target to be detected includes at least one of the treatment couch, a patient on the treatment couch, a head positioning device for fixing the patient's head and a body positioning device for fixing a patient's body; and the target component is a component that is fixed on the rotating frame and is capable of rotating with the rotating frame; and
determining whether there is a risk of collision between the target to be detected and the target component according to collision risk analysis data that is capable of reflecting a magnitude relationship between a first distance and a second distance, wherein the first distance is a minimum distance between each collision-prone position and a reference position of the radiotherapy equipment; and the second distance is a minimum distance between the target component and the reference position, and a distance between the reference position and the target component is relatively constant when the target component rotates around the rotation axis.

2. The method according to claim 1, wherein
the collision risk analysis data includes the first distance and the second distance; and
determining whether there is the risk of collision between the target to be detected and the target component according to the collision risk analysis data that is capable of reflecting the magnitude relationship between the first distance and the second distance, includes:
determining whether there is the risk of collision between the target to be detected and the target component according to the magnitude relationship between the first distance and the second distance.

3. The method according to claim 2, wherein
determining whether there is the risk of collision between the target to be detected and the target component according to the magnitude relationship between the first distance and the second distance, includes:
determining a distance threshold, the distance threshold being obtained according to the second distance and the distance threshold being less than the second distance;
determining that there is the risk of collision between the target to be detected and the target component in response to the first distance being equal to or greater than the distance threshold; and
determining that there is no risk of collision between the target to be detected and the target component in response to the first distance being less than the distance threshold.

4. The method according to claim 1, wherein the reference position is a reference line, and the reference line is a central line of the rotation axis; and
the target component includes a treatment head or an image device.

5. The method according to claim 1, wherein the second distance is obtained according to at least one of following parameters:
a distance from a bottom face of the target component to the reference position when the target component being located at a zero position, a width of the target component in an extending direction of the rotation axis, a swing angle of the target component in the extending direction of the rotation axis, and a distance that a front end of the treatment couch deviates from an isocenter of the radiotherapy equipment in the extending direction of the rotation axis.

6. The method according to claim 5, wherein the target to be detected includes a body bed board of the treatment couch for placing a patient's body, and the second distance X1 is:
$$X 1 = \sqrt{{r}^{2} + {( \frac{W}{2} )}^{2}} \times cos ( arctan \frac{W}{2 r} + α ) ;$$
wherein r is the distance from the bottom face of the target component to the reference position when the target component being located at the zero position; W is the width of the target component in the extending direction of the rotation axis; and a is the swing angle of the target component in the extending direction of the rotation axis.

7. The method according to claim 5, wherein the target to be detected includes a head support board of the treatment couch for placing a patient's head and the head positioning device, and the second distance X2 is:
X2=(r−Cy×sin(α))/cos(α);
wherein r is the distance from the bottom face of the target component to the reference position when the target component being located at the zero position; Cy is the distance that the front end of the treatment couch deviates from the isocenter of the radiotherapy equipment in the extending direction of the rotation axis; and α is the swing angle of the target component in the extending direction of the rotation axis.

8. The method according to claim 1, wherein the at least one collision-prone position includes at least one corner point of a cross section of the target to be detected is cut by a plane perpendicular to the rotation axis of the radiotherapy equipment.

9. The method according to claim 1, wherein obtaining the at least one collision-prone position of the target to be detected that is prone to collide with the target component in the radiotherapy equipment, includes:
obtaining the at least one collision-prone position of the target to be detected that is prone to collide with the target component according to a radiation therapy mode to be performed by the radiotherapy equipment;
wherein the radiation therapy mode is radiation therapy to head or radiation therapy to body.

10. The method according to claim 9, wherein the treatment couch includes a body bed board for placing a patient's body and a head support board for placing the patient's head; and
obtaining the at least one collision-prone position of the target to be detected that is prone to collide with the target component according to the radiation therapy mode to be performed by the radiotherapy equipment, includes:
obtaining the at least one target collision position of the body bed board of the treatment couch that is prone to collide with the target component when the radiation therapy mode is the radiotherapy to body.

11. The method according to claim 9, wherein the treatment couch includes a body bed board for placing a patient's body and a head support board for placing the patient's head; and
obtaining the at least one collision-prone position of the target to be detected that is prone to collide with the target component according to the radiation therapy mode to be performed by the radiotherapy equipment, includes:
determining whether one end of the body bed board of the treatment couch proximate to the head support board extends into a critical space when the radiation therapy mode is the radiation therapy to head, the critical space being a space formed when a radiation surface of a treatment head rotating one cycle around the rotation axis;
obtaining at least one target collision position of the head positioning device for fixing the patient's head and the head support board of the treatment couch that are prone to collide with the target component in response to determining that the end of the body bed board of the treatment couch proximate to the head support board does not extend into the critical space; and
obtaining at least one target collision position of the head positioning device, the head support board of the treatment couch, and the body bed board of the treatment couch that are prone to collide with the target component in response to determining that the end of the body bed board of the treatment couch proximate to the head support board extends into the critical space.

12. The method according to claim 1, wherein after determining that there is the risk of collision between the target to be detected and the target component, the method further comprises:
determining a safety rotation range of the target component;
determining that the target to be detected will not collide with the target component in response to a set rotation range of the target component completely belonging to the safety rotation range; and
determining that the target to be detected will collide with the target component in response to the set rotation range of the target component not completely belonging to the safety rotation range.

13. The method according to claim 12, wherein determining the safety rotation range of the target component, includes:
determining an arc range corresponding to each target collision position, wherein the at least one collision-prone position includes a plurality of collision-prone positions, the target collision position is a collision-prone position with the first distance greater than a corresponding distance threshold among the plurality of collision-prone positions, and the target component of the radiotherapy equipment does not collide with the collision-prone position when it being within the arc range corresponding to the target collision position; and the distance threshold is obtained according to the second distance, and the distance threshold is less than the second distance; and
determining that an intersection of arc ranges of all target collision positions being the safety rotation range.

14. The method according to claim 13, wherein determining the arc range corresponding to each target collision position, includes:
determining a critical circumference with the rotation axis as an axis and the distance threshold as a radius; and
determining a larger of two central angles corresponding to the target collision position as the arc range of the target collision position by constructing tangent lines to the critical circumference from the target collision position to obtain tangent points.

15. The method according to claim 1, wherein before obtaining the at least one collision-prone position of the target to be detected that is prone to collide with the target component in the radiotherapy equipment, the method further comprises:
obtaining stroke parameters of the treatment couch in different directions; and
obtaining the at least one collision-prone position of the target to be detected that is prone to collide with the target component in the radiotherapy equipment includes: obtaining at least one collision-prone position of the target to be detected that is prone to collide with the target component in the radiotherapy equipment in response to determining that a stroke parameter of the treatment couch in any direction is within a predetermined stroke range.

16. The method according to claim 1, wherein the reference position is an isocenter of the radiotherapy equipment or a central line of the rotation axis, and an extending direction of the central line is same as an extending direction of the rotation axis.

17. A device for collision detection of a radiotherapy equipment, the radiotherapy equipment including a rotating frame that is capable of rotating around a rotation axis and a treatment couch, the device comprises an obtaining device and a processing device;
wherein the obtaining device is configured to obtain at least one collision-prone position of a target to be detected that is prone to collide with a target component in the radiotherapy equipment; the target to be detected includes at least one of the treatment couch, a patient on the treatment couch, a head positioning device for fixing the patient's head, and a body positioning device for fixing the patient's body; and the target component is a component that is fixed on the rotating frame and is capable of rotating with the rotating frame; and
the processing device is configured to determine whether there is a risk of collision between the target to be detected and the target component according to collision risk analysis data that is capable of reflecting a magnitude relationship between a first distance and a second distance; the first distance is a minimum distance between each collision-prone position and a reference position of the radiotherapy equipment; the second distance is a minimum distance between the target component and the reference position; and a distance between the reference position and the target component is relatively constant when the target component rotates around the rotation axis.

18. A device for collision detection of a radiotherapy equipment, the device comprises a memory and a processor, wherein the memory is used to store computer-executable instructions, and the processor is connected to the memory; and the processor executes the computer-executable instructions stored in the memory when the device for collision detection of the radiotherapy equipment is running, so that the radiotherapy equipment performing one or more steps of the method for collision detection of the radiotherapy equipment according to claim 1.

19. A non-transitory computer-readable storage medium, wherein the non-transitory computer-readable storage medium has stored thereon computer-executable instructions, and one or more steps of the method for collision detection of the radiotherapy equipment according to claim 1 are implemented when a computer executes the computer-executable instructions.

20. A computer program product for collision detection of a radiotherapy equipment, wherein the computer program product is stored on a non-transitory computer-readable medium, and the computer program product is configured to perform one or more steps of the method for collision detection of the radiotherapy equipment according to claim 1 when running on a computer.
